SEQUENCE LISTING (4 sequences)

SEQ ID NO: 1 - protein, 1084 aa, danio rerio
MSDSEDSDFSDNQSERSSEAEEVEENEEEEEQGSVAGSDKAEEEGEDLEDEEEYDEEEEEDDDRPRKKARHGGFILDEADVDDEYEDEDPWEDGAEDILEKEEAEVSNLDHVVLDEDHSGSRRLQNLWRDSREEALGEYYMRKYAKSSGGEHFYGGSEDLSDDITQQQLLPGVKDPNLWTVKCKIGEERATAISLMRKFVAYQCTDTPLQIKSVVAPEHVKGYIYVEAYKQTHVKAAIEGVGNLRMGFWNQQMVPIKEMTDVLKVVKEVTNLKPKSWVRLKRGLYKDDIAQVDYVEPSQNTISLKMIPRIDLDRIKARMSMKDWFAKRKKFKRPPQRLFDAEKIRSLGGEVSHDGDFMIFEANRYSRKGFLFKSFAMSAVITEGVKPTLSELEKFEDQPEGIDLEVVTETTGKEREHNLQAGDNVEVCEGELINLQGKILSVDGNKITIMPKHEDLKDPLEFPAHELRKYFRMGDHVKVIAGRYEGDTGLIVRVEENFVILFSDLTMHELKVLPRDLQLCSETASGVDAGGQHEWGELVQLDPQTVGVIVRLERETFQVLNMHGKVLTVRHQAVNRRKDNRFAVALDSEQNNIHVKDIVKVIDGPHSGREGEIRHIFRGFAFLHCKKLVENGGMFVCKARHLVLAGGSKPRDVTNFTVGGFAPMSPRISSPMHPGGGGQPQRGGGGGGGGGMGRGRGRRDNDLIGQTVRISQGPYKGYIGVVKDATESTARVELHSTCQTISVDRQRLTTVGGKERQGRSSTHLRTPMYGSQTPIYGTGSRTPMYGSQTPLHDGSRTPHYGSQTPLHDGSRTPGQSGAWDPNNPNTPSRPDDEYEFAYDDEPSPSPQGYGGTPNPQTPGYPEVPSPQVNPQYNPQTPGTPAMYNTDQYSPYAAPSPQGSYQPSPSPQSYHQVAPSPVGYQNTHSPASYHPTPSPMAYQASPSPSPVGYSPMTPGAPSPGGYNPHTPGSNIDQASNDWVTTDIMVRVKDTFLDGGVINQTGIIRSVTGGMCSVFLQDTEKVVSISSEHLEPVTPTKNNKVKVILGEDREATGVLLSIDGEDGIVRMELDEQLKILNLRFLGKLEV

SEQ ID NO: 2 - DNA, 3888 bp, danio rerio
cacgaaaggaaaacattggccttcgctgccaggttgaagcgaacaagacagctgttaaaagtgtttccgtctggaaaacagaaaaagaaaaaacaaagagatgtcagacagcgaggacagtgatttctctgataatcagagtgagaggagcagcgaggctgaagaggtggaggagaatgaggaagaggaagaacagggaagtgtagccggcagtgacaaggctgaggaagagggagaggatttggaagatgaggaggaatatgatgaagaggaagaggaggatgacgaccgacctcggaagaaagctcgacatggtggcttcatcttggatgaagccgatgttgatgatgagtatgaagatgaagatccatgggaggatggtgctgaagacatcttagaaaaagaagaggctgaagtgtccaaccttgaccatgtagttctggatgaggatcactcgggttcaaggaggttgcagaatctctggagagattctcgtgaggaagctctgggggagtattacatgaggaaatatgccaagtcttcaggaggcgaacacttctatggtggctctgaggatctttctgatgacatcacacagcaacaattgcttcctggggtcaaggatcctaatttgtggactgttaagtgtaagattggagaggagagagcaactgcaatttccttgatgcgaaaattcgttgcttatcagtgcactgacacgccacttcaaatcaaatcagtagttgcccctgaacatgtcaaaggttatatctatgtggaggcgtacaagcagacccatgtcaaggctgccattgagggtgtgggaaacctgcgcatgggcttctggaaccagcagatggtgcccattaaagagatgacagatgtgctgaaagtggttaaggaagttactaaccttaaacccaagtcttgggttcgtctcaagagaggcctttataaagatgacattgcccaggtggactatgttgaacctagtcagaacacgatttcgttgaaaatgattcctcgtattgacctggatcgtatcaaagcaaggatgagtatgaaagactggtttgcaaagagaaagaagttcaagaggcccccacagaggctttttgatgctgaaaagatcaggtctcttggtggtgaggtcagccatgatggtgatttcatgatatttgaagcgaaccgctacagcagaaagggtttcctgttcaaaagctttgccatgtctgctgtgatcacagaaggggtgaagcccactctgtccgagctggagaagtttgaagaccagccagagggcattgaccttgaagtagtcacagagacaacaggtaaggagcgagaacacaatctccaggcgggagataatgtggaggtgtgtgaaggggagttgatcaaccttcagggcaaaatcttgagtgtggatggcaacaaaatcaccatcatgcccaagcacgaggacctcaaggatcctcttgagtttccagctcatgagttgcggaagtattttcgtatgggtgaccatgtgaaggtcatcgcggggcgttatgaaggggatacaggcctcatagttcgcgtggaggagaactttgtcattctgttctctgatctcaccatgcacgagttgaaagttttgccccgggacctgcagctgtgctctgagacggcttcaggtgtggatgctggaggccagcatgagtggggggagctggtacagttagaccctcaaactgtaggtgtcattgtcaggctggagagagagacctttcaggttctgaacatgcatgggaaagttttaacagtacggcaccaggcagtgaacaggcggaaggacaaccggtttgctgttgctctcgactctgagcaaaacaacatccatgttaaggatattgtgaaagtcattgatggacctcattcgggccgtgaaggtgaaattcgccacatcttcagaggctttgcttttcttcactgcaagaagctggttgaaaacggaggcatgtttgtttgtaaagcacgtcaccttgtactggcaggtggctctaagcccagagatgtgaccaattttacagtgggtggctttgctcccatgagcccacgcatcagcagcccaatgcaccctggaggaggtggtcagccgcaaagaggtggaggtggtggtggtggtggtggcatgggccgaggccgtggacgacgagacaatgatctcattggccagactgttcgtatatcccagggaccatacaaaggctacattggtgtggtgaaggatgccacagaatccaccgctagggtagagcttcactcaacctgccagaccatctctgtggatcgccagcgccttacaactgttggtggcaaagaaagacaaggaagaagttccacccatctacgaaccccgatgtatggttcccagacacccatttatgggactggctcacgtactcccatgtatggtagccagacacccctccatgatggaagtcgtactcctcactatggctcacagactcctcttcatgatgggagcaggacacctggccaaagtggtgcgtgggaccccaacaatccaaacacaccatcacggcctgatgacgagtatgagtttgcctacgatgacgagccctcgccgtctcctcaaggctatggtgggacacccaaccctcagacgcctggatatccagaagtgccctctccgcaggtcaaccctcagtataacccacagactcctgggactcctgccatgtacaacacagatcaatactccccatatgcagcaccttcaccccagggctcctaccagcccagtccaagtccacagagttaccaccaagtggcgccaagcccggtgggataccaaaacacacactcgcctgccagttaccaccccactccatcacccatggcttaccaggccagtccaagtcccagtccggtagggtatagtcccatgactcctggcgctccgtctcctgggggttacaaccctcacacccctggctccaacattgaccaagcctccaatgactgggtcaccacagacatcatggtgcgtgtgaaagacacattccttgatggaggcgtcatcaatcagaccggcatcatccgcagtgtcaccggaggaatgtgctctgtttttctgcaagacactgaaaaggttgttagcatttcaagcgaacacttggagcctgttacaccaaccaaaaacaataaggtgaaggtgattctgggagaggacagggaggccacaggagtcctgcttagtatcgatggtgaagacggcattgttagaatggagttggacgagcagctgaagattctcaatctccgcttccttgggaaactggaagtgtgaagaaccccacacattaaaacacgtttttctaaatgaatgcagtttgctgcagaatagtcactttccgtgagctttttttttttttttttaattagtggaaggttttagtgggtagtcttctttttctgttttctctgtggtgtagaaacgttttttggcaaattgattccacgtcaaataaaatgtgaaccgtgagttcctaaatccatccatcctgagtgttcgcatgaaatatttcatagctgctggctgttccttctgaaacttttactgcctgcccaaacttggaacatcgatcgtgaagaggaattccattgttatgctcttacgagtgattcgagctccagcagctgagataaaactgctgttcccagcgtcattaaagattctccagcatcatggtccattactttctcaacctttaatgtcattgatagttgatgtacattctgactgagcacattaaaggtttttcaaaaccctttcaaaaaaaaaaaaaaaaaaaaaaaaaaaaaaaaaaaaaaaaaaaaaaa

SEQ ID NO: 3 - protein, 1084 aa, Artificial Sequence
  foggy mutant
MSDSEDSDFSDNQSERSSEAEEVEENEEEEEQGSVAGSDKAEEEGEDLEDEEEYDEEEEEDDDRPRKKARHGGFILDEADVDDEYEDEDPWEDGAEDILEKEEAEVSNLDHVVLDEDHSGSRRLQNLWRDSREEALGEYYMRKYAKSSGGEHFYGGSEDLSDDITQQQLLPGVKDPNLWTVKCKIGEERATAISLMRKFVAYQCTDTPLQIKSVVAPEHVKGYIYVEAYKQTHVKAAIEGVGNLRMGFWNQQMVPIKEMTDVLKVVKEVTNLKPKSWVRLKRGLYKDDIAQVDYVEPSQNTISLKMIPRIDLDRIKARMSMKDWFAKRKKFKRPPQRLFDAEKIRSLGGEVSHDGDFMIFEANRYSRKGFLFKSFAMSAVITEGVKPTLSELEKFEDQPEGIDLEVVTETTGKEREHNLQAGDNVEVCEGELINLQGKILSVDGNKITIMPKHEDLKDPLEFPAHELRKYFRMGDHVKVIAGRYEGDTGLIVRVEENFVILFSDLTMHELKVLPRDLQLCSETASGVDAGGQHEWGELVQLDPQTVGVIVRLERETFQVLNMHGKVLTVRHQAVNRRKDNRFAVALDSEQNNIHVKDIVKVIDGPHSGREGEIRHIFRGFAFLHCKKLVENGGMFVCKARHLVLAGGSKPRDVTNFTVGGFAPMSPRISSPMHPGGGGQPQRGGGGGGGGGMGRGRGRRDNDLIGQTVRISQGPYKGYIGVVKDATESTARVELHSTCQTISVDRQRLTTVGGKERQGRSSTHLRTPMYGSQTPIYGTGSRTPMYGSQTPLHDGSRTPHYGSQTPLHDGSRTPGQSGAWDPNNPNTPSRPDDEYEFAYDDEPSPSPQGYGGTPNPQTPGYPEVPSPQVNPQYNPQTPGTPAMYNTDQYSPYAAPSPQGSYQPSPSPQSYHQVAPSPVGYQNTHSPASYHPTPSPMAYQASPSPSPVGYSPMTPGAPSPGGYNPHTPGSNIDQASNDWVTTDIMVRVKDTFLDGGVINQTGIIRSVTGGMCSDFLQDTEKVVSISSEHLEPVTPTKNNKVKVILGEDREATGVLLSIDGEDGIVRMELDEQLKILNLRFLGKLEV

SEQ ID NO: 4 - DNA, 3888 bp, Artificial Sequence
  foggy mutant
cacgaaaggaaaacattggccttcgctgccaggttgaagcgaacaagacagctgttaaaagtgtttccgtctggaaaacagaaaaagaaaaaacaaagagatgtcagacagcgaggacagtgatttctctgataatcagagtgagaggagcagcgaggctgaagaggtggaggagaatgaggaagaggaagaacagggaagtgtagccggcagtgacaaggctgaggaagagggagaggatttggaagatgaggaggaatatgatgaagaggaagaggaggatgacgaccgacctcggaagaaagctcgacatggtggcttcatcttggatgaagccgatgttgatgatgagtatgaagatgaagatccatgggaggatggtgctgaagacatcttagaaaaagaagaggctgaagtgtccaaccttgaccatgtagttctggatgaggatcactcgggttcaaggaggttgcagaatctctggagagattctcgtgaggaagctctgggggagtattacatgaggaaatatgccaagtcttcaggaggcgaacacttctatggtggctctgaggatctttctgatgacatcacacagcaacaattgcttcctggggtcaaggatcctaatttgtggactgttaagtgtaagattggagaggagagagcaactgcaatttccttgatgcgaaaattcgttgcttatcagtgcactgacacgccacttcaaatcaaatcagtagttgcccctgaacatgtcaaaggttatatctatgtggaggcgtacaagcagacccatgtcaaggctgccattgagggtgtgggaaacctgcgcatgggcttctggaaccagcagatggtgcccattaaagagatgacagatgtgctgaaagtggttaaggaagttactaaccttaaacccaagtcttgggttcgtctcaagagaggcctttataaagatgacattgcccaggtggactatgttgaacctagtcagaacacgatttcgttgaaaatgattcctcgtattgacctggatcgtatcaaagcaaggatgagtatgaaagactggtttgcaaagagaaagaagttcaagaggcccccacagaggctttttgatgctgaaaagatcaggtctcttggtggtgaggtcagccatgatggtgatttcatgatatttgaagcgaaccgctacagcagaaagggtttcctgttcaaaagctttgccatgtctgctgtgatcacagaaggggtgaagcccactctgtccgagctggagaagtttgaagaccagccagagggcattgaccttgaagtagtcacagagacaacaggtaaggagcgagaacacaatctccaggcgggagataatgtggaggtgtgtgaaggggagttgatcaaccttcagggcaaaatcttgagtgtggatggcaacaaaatcaccatcatgcccaagcacgaggacctcaaggatcctcttgagtttccagctcatgagttgcggaagtattttcgtatgggtgaccatgtgaaggtcatcgcggggcgttatgaaggggatacaggcctcatagttcgcgtggaggagaactttgtcattctgttctctgatctcaccatgcacgagttgaaagttttgccccgggacctgcagctgtgctctgagacggcttcaggtgtggatgctggaggccagcatgagtggggggagctggtacagttagaccctcaaactgtaggtgtcattgtcaggctggagagagagacctttcaggttctgaacatgcatgggaaagttttaacagtacggcaccaggcagtgaacaggcggaaggacaaccggtttgctgttgctctcgactctgagcaaaacaacatccatgttaaggatattgtgaaagtcattgatggacctcattcgggccgtgaaggtgaaattcgccacatcttcagaggctttgcttttcttcactgcaagaagctggttgaaaacggaggcatgtttgtttgtaaagcacgtcaccttgtactggcaggtggctctaagcccagagatgtgaccaattttacagtgggtggctttgctcccatgagcccacgcatcagcagcccaatgcaccctggaggaggtggtcagccgcaaagaggtggaggtggtggtggtggtggtggcatgggccgaggccgtggacgacgagacaatgatctcattggccagactgttcgtatatcccagggaccatacaaaggctacattggtgtggtgaaggatgccacagaatccaccgctagggtagagcttcactcaacctgccagaccatctctgtggatcgccagcgccttacaactgttggtggcaaagaaagacaaggaagaagttccacccatctacgaaccccgatgtatggttcccagacacccatttatgggactggctcacgtactcccatgtatggtagccagacacccctccatgatggaagtcgtactcctcactatggctcacagactcctcttcatgatgggagcaggacacctggccaaagtggtgcgtgggaccccaacaatccaaacacaccatcacggcctgatgacgagtatgagtttgcctacgatgacgagccctcgccgtctcctcaaggctatggtgggacacccaaccctcagacgcctggatatccagaagtgccctctccgcaggtcaaccctcagtataacccacagactcctgggactcctgccatgtacaacacagatcaatactccccatatgcagcaccttcaccccagggctcctaccagcccagtccaagtccacagagttaccaccaagtggcgccaagcccggtgggataccaaaacacacactcgcctgccagttaccaccccactccatcacccatggcttaccaggccagtccaagtcccagtccggtagggtatagtcccatgactcctggcgctccgtctcctgggggttacaaccctcacacccctggctccaacattgaccaagcctccaatgactgggtcaccacagacatcatggtgcgtgtgaaagacacattccttgatggaggcgtcatcaatcagaccggcatcatccgcagtgtcaccggaggaatgtgctctgattttctgcaagacactgaaaaggttgttagcatttcaagcgaacacttggagcctgttacaccaaccaaaaacaataaggtgaaggtgattctgggagaggacagggaggccacaggagtcctgcttagtatcgatggtgaagacggcattgttagaatggagttggacgagcagctgaagattctcaatctccgcttccttgggaaactggaagtgtgaagaaccccacacattaaaacacgtttttctaaatgaatgcagtttgctgcagaatagtcactttccgtgagctttttttttttttttttaattagtggaaggttttagtgggtagtcttctttttctgttttctctgtggtgtagaaacgttttttggcaaattgattccacgtcaaataaaatgtgaaccgtgagttcctaaatccatccatcctgagtgttcgcatgaaatatttcatagctgctggctgttccttctgaaacttttactgcctgcccaaacttggaacatcgatcgtgaagaggaattccattgttatgctcttacgagtgattcgagctccagcagctgagataaaactgctgttcccagcgtcattaaagattctccagcatcatggtccattactttctcaacctttaatgtcattgatagttgatgtacattctgactgagcacattaaaggtttttcaaaaccctttcaaaaaaaaaaaaaaaaaaaaaaaaaaaaaaaaaaaaaaaaaaaaaaa

(19) United States
(12) Patent Application Publication
(10) Pub. No.: US 2004/0197900 A1
(43) Pub. Date: Oct. 7, 2004

(54) Foggy

(76) Inventors: Arnon Rosenthal, Woodside, CA (US); Su Guo, San Francisco, CA (US)

Correspondence Address: Attn: Craig G. Svoboda, Genentech, Inc., 1 DNA Way, M.S. 49, South San Francisco, CA, 94080, US

(21) Appl. No.: 10/407,800

(22) Filed: Apr. 4, 2003

(51) Int. Cl. C12N 5/08; A61K 38/17

(52) U.S. Cl. 435/368; 514/12

(57) ABSTRACT

The present invention relates to neucleotide and amino acid sequences having homology to the zebrafish transcription elongation factor foggy, and to neuronal formation, to a method of directing the differentiation of neuroprogenitor cells into dopaminergic or serotonergic neurons and to a method of treating disorders characterized by abnormalites in and the activity of dopaminergic and serotonergic neurons.

### FIELD OF THE INVENTION

The present invention relates to nucleotitde and amino acid sequences having homology to the zebrafish transcription elongaton factor foggy and to neuronal formation and methods of treating disorders characterized by abnormalities in and the activity of dopaminergic (DA) and serotonergic (5HT) neurons.

### BACKGROUND OF THE INVENTION

During development of the animal nervous system, precursors with small variability in position, birth date or distribution of cytoplasmic components differentially respond to localized extrinsic signals and give rise to hundreds of distinct classes of neurons and non-neuronal cells. Both the initial difference among progenitors and their final phenotypes appear to be dictated in part by unique temporal or cytological changes. For example, nested expression of Hox genes anticipates segmentation along the anterior-posterior axis of the vertebrate hindbrain. Lumsden and Krumlauf, 1996, Science 274: 1109-1114, whereas unique combinations of Lim, Islet and Lim-HD transcription factors precedes and predicts subtypes of the vertebrate motoneurons and their future axonal trajectories. Tsuchidaetal., 1994, Cell 79: 957-70; Sharma et al., 1998, Cell95: 817-28. The differential response to extra-cellular factors is also specified by the history of gene expression and subsequently translates into unique patterns of expressed genes. Thus, the response to hedgehog (Hh) in the neural plate required the presence of the Gli family of transcription factors (Ruiz i Altaba, 1997, Cell 90: 193-6) and different concentrations of Hh elicit distinct transcriptional responses in phenotypically homogeneous group of cells. Tanabe and Jessell, 1996, Science 274: 1115-1123. Likewise, the response of sensory organ precursors (SOP) lineage to the Notch-ligand Delta in the fly is affected by asymmetric distribution of the protein Numb and Prospero and is translated into distinct daughter cell fates. Artavanis-Tsakonas et al., 1999, Science 284: 770-776; January and January 1998, Nature 392: 775-78. Although in rare cases the pattern of gene expression is influenced by maternal inheritance of mRNA or gene rearrangement, the expression of most genes appears to be regulated at the transcriptional level.

To date, the key regulatory step in gene expression is thought to be the transcription initiation phase. Thus, studies in the Drosophila embryo had demonstrated that graded intracellular signals are carried by transcription initiation factors and that genes differ in their response because their promoters vary in the different types, number of affinity of binding sites that they carry. Burz et al., 1998, EMBO J 17: 5998-6009; Jiang and Levine, 1993, Cell 72: 741-52. Similarly, the unique transcriptional response to the extrinsic signals Hh, Wnt (Eastman and Grosschedi, 1999, Curr. Opin. Cell Biol. 11: 233-40), BMP (Massague, 1998, Ann. Rev. Biochemistry 67: 753-91), Delta/Serrate (Artavanis-Tsakonas et al., supra) and retinoic acid, is attributed to the use of distinct transcription initiation factors with different distribution of promoter binding sites. However, the production of translatable mRNA is a complex process amenable to regulation at multiple steps. For example, a relatively neglected area with regard to the control of spatial and temporal gene expression during development in vertebrates is the mRNA elongation phase. Greenblatt, 1997, Curr. Opin. Cell Biol. 9: 310-9; Shilatifard, 1998, FASEB J. 12: 1437-46; Uptain et al., 1997, Ann. Rev. Biochem. 66: 117-72. It is known that several eukaryotic viruses including adenovirus, vaccinia and human immunodeficiency virus (HIV) use transcription elongation to coordinate the expression of early and late genes and to adjust replication to the physiological status of their host. Garber and Jones, 1999, Curr. Opin. Immunol. 11: 460-65; Kim et al., 1999, Mol. Cell. Biol. 19: 5960-68; Wu-Baer et al 1998, J. Mol. Biol. 277: 179-97. In addition, there are indications that in cell culture, the expression of some vertebrate genes including the protooncogenes, c-fins, c-fos, c-myb and c-myc, are regulated in part at the transcription elongation step. Uptain et al., supra. Additional support for the possible importance of regulation of transcription elongation stem from the finding that the Hipple-Lindau tumor suppressor gene product binds to transcription elongation factors (Duan et al., 1995, Science 269: 1402-6), and that the myeloid-lymphoid leukemia gene is often fused with another elongation factor. Shilatifard, supra. Likewise, the growth retardation, high incidence of cancers and/or premature aging, which typify Bloom's and Werner's syndromes, are caused by mutations in DNA helicase that appear to be essential also for elongation by RNA Polymerase (Pol) I. Gray et al., 1997, Nature Genetics 17: 100-3.

Biochemical studies using in vitro systems further revealed the presence of over a dozen different protein complexes, which exert stimulatory or inhibitory influences on transcription elongation. Greenblatt, supra. These include stimulatory factors that enable Pol II to ignore or escape transient pauses or permanent arrests in transcription elongation inhibitors 5,6-dichloro-1-beta-D-ribofuranosylbenzimidazole (DRB) and H8. Wada et al., 1998, Genes & Devel. 12: 343-56. Subsequently, this complex was shown to be composed of 14 Kd and 160 Kd nuclear proteins (Wada et al., supra), which are structurally homologous to the Saccharomyces cerevisiae transcription elongation factors Spt4 and 5 (SuPpressors of Ty insertion mutation). Hartzog et al., 1998, Genes & Devel. 12: 357-69; Swanson et al., 1991, Mol. Cell. Biol. 11: 3009-19; Swanson and Winston, 1992, Genetics 132: 325-36. In vitro, DSIF inhibits the elongation activity of hypophosphorylated Pol II, following synthesis of 30-60 nucleotides, in part through physical interaction with the negative elongation factor (NELF) complex (Yamaguchi et al., 1998, Genes Cells 3: 9-15) and the large subunit of Pol II. Yamaguchi et al., 1999, J. Biol. Chem. 274: 8085-92. The inhibitory action of Spt5/DSIF is not constitutive but instead appears to be regulated by post-translational modifications. For example, phosphorylation of Pol II CTD by the DRB and H8-sensitive P-TEFb kinase that is composed of CDK9 and cyclin T was shown to facilitate the release of DSIF and NELF from the elongation complex. Wada et al., 1998, EMBO J. 17: 7395-403; Yamaguchi et al., 1998, supra. In addition, under low concentrations of ribonucleotides, the DSIF complex acts as a stimulator rather than inhibitor of elongation in vitro. Wada et al., 1998, supra. It also acts as a positive elongation factor for the HIV transcripts (Kim et al., supra; Wu-Baer et al., supra), supporting the notion that it is a versatile regulator of elongation. Taken together, these studies provided evidence that vertebrates possess the machinery to regulated transcription elongation but the physiological function, prevalence and significance of such regulation in vivo remains to be elucidated

Serotonergic neurons are important in the regulation of food intake, hormone secretion, responses to stress, pain and immune function. Serotonergic neurons innervate nearly every area of the central nervous system, including the cerebral cortex, limbic system and spinal cord, and can influence multiple functions of the brain, such as behaviors, appetite, pain, sexual activity, cardiovascular function, hormone secretion, and temperature regulation. Serotonergic dysfunction likely plays a role in the pathophysiology of various psychiatric, neurologic and neuron-related other diseases. For example, mental depression, Asberg et al., J. Clin. Psychiatry 47(4): 23-35 (1986); suicide, Asberg et al., supra, Lester, D. Pharmocopsychiatry 28 (2): 45-50 (1995), schizophrenia and violent aggressive behavior, Brown et al., J. Clin. Psychiatry 54(4): 31-41(1990), Eichelman, B. S., Annu. Rev. Med. 41: 149-158 (1990), Jacobs and Gelperin (1981) Serotonin Neurotransmission and Behavior, The MIT Press, Cambridge, Mass. Serotonin uptake inhibitors have been used in the treatment of mental depression, obsessive-compulsive disorder and bulimia. Fuller, R. W., “Serotonin uptake inhibiors: Uses in clinical therapy and in laboratory research,” Progress in Drug Research 45: 167-204, Birkhäuser-Varlag, Basel (1995). As serotonergic neurons innervate cerebral blood flow, serotonin receptor agonists, such as Sumatriptan, have been employed to abort migraine attacks. Plosker, G.L. et al., Drugs 94(4): 622-651 (1994). Most of the known and cloned serotonin receptors belong to a G-protein coupled superfamily of receptors having seven membrane-spanning domains. Hoyer et al., Pharmacol. Rev. 46(2): 157-203 (1994). Some serotonin receptor subtypes couple negatively to adenylate cyclase, while others couple positively, while other are coupled to activation of phospholipase C, or ligand-gated ion channels. Fuller, R. W., Ann. N.Y. Acad. Sci. 780: 176-184 (1996).

Dopaminergic neurons control movement and reward-associated behaviors. These neurons innervate multiple structures in the forebrain, and their degneration or abnormal funciton is associated with Parkinson's disease, schizophrenia and drug addiction. Hynes et al., Cell 80: 95-101 (1995). Dopaminergic neurons located in the substantia nigra have a great impact upon striatal activity as bilateral lesions of the nigrostriatal pathway produce a syndrome in experimental animals that is quite similar to the observed motor dysfunctions observed in Parkinson's disease: resting tremor, regidity, akinesia and postural abnormalities. Bilateral lesions of the nigrostriatal pathway caused by 6-hydroxydopamine (OHDA) caused profound akinesia, adipsia, aphagia and sensory neglect in rodents, Ungerstedt, U., Acta Physiol. Scand. 1971 (Suppl. 367): 95-121; Yirek and Sladek, 1990, Annu. Rev. Neurosci. 13: 415-440.

Loss of striatal DA is associated with an alternation in the number of target receptors located on striatal cells. In parkinsonism, changes in the status of DA receptors may be dependent on the stage of progression of the disease. The hallmark of parkinsonism is a severe reduction of dopamine in all components of the basal ganglia, Hornykiewicz, O., 1988, Mt. Sinai J. Med. 55: 11-20. Dopamine and its metabolites are depleted in the caudate nucleus, putamen, globus pallidus and pars compacta of the substantia nigra. Moderate losses of DA are found in the nucleus accumbens, lateral hypothalamus, medial olfactory region, and amygdaloid accumbens. Changes in non dopaminergic neuronal systems include decreases in tissue concentrations of norepinephrine, serotonin, substance P, neurotension and several neuropeptides in most basal gangliar structure, cerebellar cortex, and spinal cord.

Schizophrenia is often characterized by peculiar thought disorders, a disturbance of emotional or affective responses to the environment and autism—a withdrawal from interactions with other people. Hallucinations have also been associated as symptomatic of schizophrenia. Phenothiazine drugs are generally acknowledged to be effective in alleviating the symptoms of schizophrenia. Other medications have involved neurotransmitters. Snyder et al., Science 184: 1243-1253 (1974). Extended use of and toxic doses of amphetamines also elicit schizophrenic-like symptoms.

Considerable attention has been placed on neural transplantation in patients afflicted with Parkinson's disease. These clinical experiments essentially evolved from basic scientific research using various animal models of parkinsonism as recipients of either fetal embryonic nerve cell or paraneuronal tissue grafts to brain-damaged areas. While the concept for neural transplantation is quite old, major advances have occurred only within the last two decades, and many issues remain such as the potential long-term effectiveness of neural grafts to restore and maintain normalized function in animal models of a variety of disorders. Animal experimentation with fetal DA nerve cell grafts have provided encouragement that such grafts could reverse DA deficits and restore motor function in animals with experimental lesions of the nigrostriatal DA system. However numerous ethical, legal and safety issues are coincident with the use of fetal tissue in clinical research, factors which have only exacerbated an already limited supply, all of which establishes an urgent need for alternative sources of dopaminergic neurons.

### SUMMARY OF THE INVENTION

The present invention provides generally for the nucleotide, amino acid sequence and biochemical characterization of foggy, a transcription elongation factor which is essential for proper neuronal development. Mutant organisms producing defective foggy polypeptide, while at first glance appear to be morphologically normal, upon closer inspection show deficits in hypothalamic and retinal dopaminergic (DA) neurons, hindbrain noradrenergic (NA) neurons and the neural crest-derived sympathetic (NA) neurons. Interestingly, the foggy mutants, while deficient in hypothalamic DA neurons in the foggy mutant, were surprisingly also characterized by an increase in the number of neighboring serotonergic (5HT) neurons. These foggy mutants also showed defects in neural development in the developing retina, where deficits in amacrine and photoreceptor neurons were observed. In contrast, the retinal ganglion neurons were not defective, and even exhibited a small increase in numbers.

In one embodiment, the present invention provides a method for forming dopaminergic neurons comprising contacting neuroprogenitor cells with an effective amount of a foggy polypeptide. In one aspect, the foggy polypeptide comprises SEQ ID NO:1 of FIG. 11. In a specific aspect, said contacting occurs in vitro.

In another embodiment, the present invention provides a method for forming serotonergic neurons comprising contacting neuroprogenitor cells with and effective amount of a foggy polypeptide antagonist. In one aspect, the foggy polypeptide antagonist comprises SEQ ID NO:3. In a specific aspect, said contacting occurs in vitro.

In another embodiment, the invention provides a method for treating a disorder in a mammal wherein said disorder is characterized by the degeneration of dopaminergic neurons comprising transplanting into said mammal, a therapeutically effective amount of neuroprogenitor cells pretreated with an effective amount of a foggy polypeptide. In another embodiment, the invention provides a method of treating a disorder in a mammal, wherein said disorder is characterized by the degeneration of serotonergic neurons, said method comprising transplanting into said mammal neuroprogenitor cells pretreated with an effective amount of a foggy polypeptide antagonist.

In another embodiment, the invention provides a method of using serotonergic neurons resulting from the application of a foggy polypeptide to neuroprogentor cells for the treatment of disorders that are characterized by an abnormal regulation of food intake, hormone secretion, stress response, pain and immune function, sexual activity, cardiovascular function and/or temperature regulation. In a specific aspect, such disorders include various psychiatric, neurologic and other diseases, e.g., mental depression, suicidal feelings, violent aggressive behavior, obsessive-compulsive behavior, anorexia/bulimia and schizophrenia.

In another embodiment, the invention provides a method for using dopaminergic neurons resulting from the application of a foggy polypeptide antagonist to neuroprogentor cells for the treatment of disorders that are characterized by abnormalities in the regulation of postural reflexes, movement and/or reward-associated behaviors. In a specific aspect, such disorders include Parkinson's disease, schizophrenia and drug addiction. Alternatively, such disorders may result from lesions due to trauma or other illness which results in Parkinson's like conditions such as resting tremor, rigidity, akinesia and postural abnormality, including akinesia, adipsia, aphagia and sensory neglect.

In another embodiment, the invention provides a method of coadministering one or more neuronal survival factors to a mammal in combination with a foggy polypeptide or foggy polypeptide antagonist for the treatment of a neurological disorder. In a specific aspect, the coadministration of the neuronal survival factor occurs before, after or concurrent with the administration of the foggy polypeptide or antagonist. In another aspect, the neuronal survival factors may also be administered with the transplantation of the neuroprogenitor cells. In another specific aspect, the neuronal survival agent may be nerve growth factor (NGF), ciliary neurotrophic factor (CNTF), brain derived neurotrophic factor (BDNF), neurotrophin-3 (NT-3), neurotrophin-4 (NT-4), FGF-x, IL-1β:, TNF-α, insulin-like growth factor (IGF-1, IGF-2), transforming growth factor beta (TGF-β, TGF-β1) or skeletal muscle extract.

In yet another embodiment, the invention provides composition of matter comprising neuroprogenitor cells and an effective amount of a foggy polypeptide or a foggy polypeptide antagonist.

In yet another embodiment, the invention provides a medical device comprising neuroprogenitor cells and a means for releasing an effective amount of a foggy polypeptide or a foggy polypeptide antagonist to stimulate differentiation into serotonergic or dopaminergic neurons, respectively.

In yet another embodiment, the invention provide a composition comprising a pharmaceutically-acceptable carrier and an effective amount of a foggy polypeptide or foggy polypeptide antagonist to stimulate differentiation of neuroprogenitor cells into serotonergic neurons or dopaminergic neurons, respectively.

In yet another embodiment, the invention provides an isolated nucleic acid molecule that encodes a foggy polypeptide or a foggy polypeptide antagonist.

In one aspect, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule encoding a foggy polypeptide or foggy polypeptide antagonist having a full-length amino acid sequence as disclosed herein, or any other specifically defined fragment of the full-length amino acid sequence as disclosed herein, or (b) the complement of the DNA molecule of (a).

In one aspect, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule comprising the coding sequence of a full length foggy polypeptide or foggy polypeptide antagonist cDNA as disclosed herein, or any other specifically defined fragment of the full-length amino acid sequence as disclosed herein, or (b) the complement of the DNA molecule of (a).

In another embodiment, the invention provides isolated foggy polypeptide or foggy polypeptide antagonists encoded by any of the isolated nucleic acid sequences hereinabove identified.

In a certain aspect, the invention provides an isolated foggy polypeptide or foggy polypeptide antagonist, comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a foggy polypeptide or foggy polypeptide antagonist having a full-length amino acid sequence as disclosed herein, or any other specifically defined fragment of the full-length amino acid sequence as disclosed herein.

In yet another embodiment, the invention provides agonists and antagonists of a native foggy polypeptide as defined herein. In a particular embodiment, the agonist or antagonist is an anti-foggy antibody or a small molecule.

In a further embodiment, the invention provides a method of identifying agonists or antagonists to a foggy polypeptide which comprise contacting the foggy polypeptide with a candidate molecule and monitoring a biological activity (e.g., transcription elongation) mediated by said foggy polypeptide. Preferably, the foggy polypeptide is a native foggy polypeptide.

In other embodiments, the invention provides chimeric molecules comprising any of the herein described polypeptides fused to a heterologous polypeptide or amino acid sequence. Example of such chimeric molecules comprise any of the herein described polypeptides fused to an epitope tag sequence or a Fc region of an immunoglobulin.

In another embodiment, the invention provides an antibody which binds, preferably specifically, to any of the above or below described polypeptides. Optionally, the antibody is a monoclonal antibody, humanized antibody, antibody fragment or single-chain antibody.

In yet other embodiments, the invention provides oligonucleotide probes which may be useful for isolating genomic and cDNA nucleotide sequences, measuring or detecting expression of an associated gene or as antisense probes, wherein those probes may be derived from any of the above or below described nucleotide sequences.

In yet other embodiments, the present invention provides methods of using the foggy polypeptides of the present invention for a variety of uses based upon the functional biological assay data presented in the Examples below.

### BRIEF DESCRIPTION OF THE DRAWINGS

FIGS. 1A-J show the foggy mutant deficient in DA neurons and a corresponding supernumerary 5HT neurons in the hypothalamus. The Left panels show WT, and right panels show foggy embryos. (A-B) Lateral views showing 20 day old foggy embryo with grossly normal morphology, reduced neural crest derived melanocytes, and blood accumulation near the heart due to block of circulation. FIGS. 1C-H are ventral views of 2-day old embryos labeled with TH antibody (1C-D), 5HT antibody (1E-F), and TH (green) and 5HT (red) double staining (1G-H) showing the reduction of TH+ DA neurons and a corresponding increase of 5HT ir neurons in the hypothalamus. Cells that express both TH and 5HT (yellow) are indicated with arrow-heads (FIG. 1H). FIGS. 1I-J are dorsal views of embryos labeled with 5HT antibody showing that hindbrain 5HT+ neurons appear normal in the foggy embryo. The increased hypothalamic 5HT+ neurons are out of the focal plane.

FIGS. 2A-L show the neuronal phenotypes in the foggy retina. WT are on the left, and foggy embryos are on the right. FIGS. 2A-B are 3-day old retina labeled with TH antibody showing that the TH+ DA amacrine neurons are missing in the foggy mutant. FIGS. 2C-F are whole mount in situ hybridization of 2-day retina with red opsin (C-D) and glutamic acid decarboxylase (GAD-67), while (E-F) shows the deficit of red opsin+ cone photoreceptors and GAD67+GABAergic amacrine neurons in the foggy mutant. FIGS. 2G-H are 2-day old retina labeled with Zn-8 antibody that recognizes ganglion neurons, showing that the ganglion neurons are not reduced, and perhaps slightly increased in the foggy mutant. FIGS. I-J are whole mount in situ hybridization of 2-day old embryos with γ-crystallin, showing that its expression is normal in the foggy mutant lens. FIGS. 2K-L are a histological analysis of eyes from 72 hpf WT (FIG. 2K) and mutant zebrafish (FIG. 2L) embryos. Eyes from mutant embryos are approximately 50 μm smaller in diameter relative to WT. All retinal mutant embryos are present and are indicated as gel (ganglion cell layer), ipl (inner plexiform layer), inl (inner nuclear layer), opl (outer plexiform layer), and pcl photoreceptor layer. Lightly basophilic cells (amacrine cells) are lacking within the inner nuclear layer of the mutant.

FIGS. 3A-L show noradrenergic LC neurons in the hindbrain, and neural crest-derived sympathetic neurons in the PNS are defective in foggy mutant embryos. On the left are WT, while the right side show the mutantfoggy embryos. FIGS. 3A-B are dorsal views of 36 hpf embryos showing that the DBH+ LC neurons are defective in the foggy mutant. FIGS. 3C-D are ventral views of 2-day old embryos labeled with TH antibody showing that the sympathetic neurons are defective in the foggy mutant. FIGS. 3E-F are lateral view of 12 somites WT and mutant embryos showing normal expression of zphox2a mRNA in LC progenitors. FIGS. 3G-H are ventral views of 2-day embryos showing the absence of zphox2a RNA from sympathetic neurons. FIGS. 3I-J are dorsal views of 36 hpf embryos labeled with 3A10 antibody, showing a normal complement of hindbrain Mauthner neurons in the foggy mutant. FIGS. 3K-L are lateral views of 2-day old embryos labeled with Hu antibody showing a normal complement of neural crest derived dorsal root ganglion (DRG) neurons in the foggy mutant.

FIGS. 4A-B identify the closely linked DNA polymorphic markers by AFLP and construction of a physical map in the foggy region. FIG. 4A is an example showing Genescan software output of PCR fragment analysis run on the automated sequencer ABI 310. Each peak represents a PCR fragment, and the unique peak indicated by the red arrow represents a PCR fragment, and the unique peak indicated by the red arrow represents a DNA marker linked to the foggy locus. FIG. 4B shows the two closely linked AFLP markers, ETACMTCT155 and ETACMGAT270, which were used as starting points to isolate large genomic DNA clones (YACs, BACs and PACs) that span the entire foggy region. The size of all TAC, BAC and PAC clones was determined by pulsed field gel electrophoresis and restriction enzyme digestion. The ends of all clones were sequenced, and the sequencing information was used to design specific PCR primers used for subsequent walk, for genetic linkage analysis by SSCP and PFLP and for confirming the genomic location by radiation hybrid panel analysis. B108J11 was found to contain the foggy gene by transformation rescue assay (see FIG. 5) and was subsequently sequenced in its entirety. It was also used as a probe to screen cDNA libraries to isolated the foggy cDNA.

FIGS. 5A-C show the rescue of the foggy mutant phenotype by the injection of BAC clones. FIG. 5A illustrates that injections of control DNA or P21917 did not rescue the foggy mutant phenotype. For example, the mutant embryo showed reduced melanocytes, blocked blood circulation, and deficits in DA and LC neurons. FIG. 5B shows that injection of B109J11 rescued the foggy mutant phenotype: out of 22 injection mutant animals, 10 were completely rescued (B, left panels) and 12 were partially rescued (B, right panels), judged by the recovery of melanocytes, blood circulation and DA and LC neurons. FIG. 5C shows that PCR genotyping of the injected embryos with tightly linked polymorphic markers flanking the foggy locu: B172K17p (0.04+/−0.02 cM from foggy), and ETACMGAT270 (0.06+/−cM from foggy). This identified that the rescued embryos were indeed genotypically mutant for foggy.

FIGS. 6A-D show the encoded amino acid sequence of the foggy polypeptide, a comparison of the Spt5/foggy family of proteins, and the mutation detection in foggy. FIG. 6A shows the deduced amino acid sequence of the zebrafish foggy, with the N-terminal acidic region underlined. The conserved regions in the center and at the C-terminus are marked with brackets. Four KOW motifs are underlined and labeled kow1-4. Two putative nuclear localization signals are boxed. Hexapeptide repeats are doubly underlined. FIG. 6B is a bar illustration showing the homology between foggy and its homologs from other species, including the acidic, N-terminal conserved central regions, hexapeptide repeats and conserved C-terminal domains. FIG. 6C shows an amino acid sequence alignment of the C-terminal domain with conserved residues boxed and invariant amino acids marked with asterisks. The foggy^{m806} mutation changes an invariant amino acid 1012-Valine to Aspartic acid. FIG. 6D is an Abi-automated sequencer produced chromatograph showing that a single nucleotide change from T→A leads to amino acid change from WT 1012-Val to mutant Asp in foggy^{m806}.

FIGS. 7A-J show the expression of foggy. The whole mount in situ hybridization with the foggy RNA probe (A-F) shows that maternal foggy mRNA is present in all blastomeres in the sphere stage embryo (A). In the tailbud stage embryo (shown in FIG. 7B), foggy mRNA is more concentrated in the neural plate, while low level expression is also detected elsewhere. During somitogenesis, in the 28 hpf embryo, foggy mRNA is highly expressed in the brain (FIG. 7C). In the 2-day old embryo, foggy mRNA is still detected more in the brain than elsewhere, but somewhat downregulated comparing to the earlier stage (FIG. 7E). The foggy mRNA expression pattern displayed no different between WT (FIGS. C,E) and foggy mutant embryos (FIGS. D,F). Mammalian Cos-7 cells transfected with FLAG-tagged WT foggy construct, and visualized by DAPI (FIG. 7G) and FLAG antibody staining (FIG. 7H) shows that WT foggy protein enters the cell nucleus. Cos-7 cells transfected with FLAG-tagged mutant foggy construct (FIGS. 7H, & 7J), shows that the mutant foggy protein is still capable of entering the cell nucleus.

FIGS. 8A-C show that the foggy mutation abolishes the negative but not the positive function of the foggy/zSpt5 in vitro. FIG. 8A shows that foggy/zSpt5 proteins used in the following assays were resolved by 7.5% SDS-PAGE and stained with Coomassie Brilliant Blue. FIG. 8B shows the depletion/add back assays using crude HeLa nuclear extract. Human Spt4 (5 ng) and either zSpt5 (50 ng), or wild-type or mutant foggy/zSpt5 (50, 150, 450 ng) were added back to the DSIF-depleted extract, and transcription reactions were allowed to initiate for 10 minutes in the presence or absence of DRB. Plasmid TF3-6C2At, which contains a 380-nt G-free cassette downstream of the adenovirus E4 promoter, was used as a template. FIG. 8C shows the depletion/add back assays which were performed using pSLG402 as a template under limiting NTP concentrations, which allows us to measure the stimulatory activity of foggy/zSpt5. As schematically drawn at the top, pSLG402 contains two G-free cassettes downstream of the adenovirus major-late promoter. The amounts of two RNA bands were quantified using a FLA2000 image analyzer (Fuji), and the promoter distal to proximal ratios were calculated.

FIGS. 9A-C show the effects of the foggy mutation on the positive function of foggy/zSpt5. Depletion/add-back assays were done using pSLG402 as a template under limiting NTP concentrations, which allowed the measurement of the stimulatory activity of foggy/zSpt5. Shown schematically in FIG. 9A, pSLG402 contains two G-free cassettes downstream of the adenovirus major-late promoter. The actual gel image in shown in FIG. 9B. The amounts of two RNA bands were quantified using a FLA2000 image analyzer (Fuji). FIG. 9C shows a calculation of the promoter distal to proximal ratios.

FIG. 10 shows the native sequence nucleic acid sequence (SEQ ID NO:2) encoding a native sequence foggy polypeptide of FIG. 11 (SEQ ID NO:1). The 3-letter start and stop codons are shown in bold font and underlined. The location of the T→A mutation is also identified in bold font and underlined.

FIG. 11 shows a native foggy amino acid sequence (SEQ ID NO:1). The location of the Val→Asp mutation at position 1012 is also identified in bold font and underlined.

### DETAILED DESCRIPTION OF THE INVENTION

I. Definitions

The terms “foggy polypeptide” and “foggy” as used herein refer to various polypeptides described herein. These terms encompass both native sequence polypeptides as well as variants thereof (which are further defined herein) and antagonists thereof. The foggy polypeptides and foggy polypeptide variants described herein may be isolated from a variety of sources, such as from human tissue types or from another source, or prepared by recombinant or synthetic methods.

A “native sequence foggy polypeptide” comprises a polypeptide having the same amino acid sequence as the corresponding foggy polypeptide derived from nature. Such native sequence foggy polypeptides can be isolated from nature or can be produced by recombinant or synthetic means. The term “native sequence foggy polypeptide” specifically encompasses naturally-occurring truncated forms, naturally-occurring variant forms (e.g., alternatively spliced forms) and naturally-occurring allelic variants of the polypeptide. In various embodiments of the invention, the native sequence foggy polypeptides disclosed herein are mature or full-length native sequence polypeptides comprising the full-length amino acids sequences shown in the accompanying figures. Start and stop codons are shown in bold font and underlined in the figures.

“Foggy polypeptide variant” or “foggy polypeptide antagonist variant” means an active foggy polypeptide as defined herein having at least about 80% amino acid sequence identity with a full-length native sequence foggy polypeptide or foggy polypeptide antagonist sequence, respectively, as disclosed herein, or any other fragment of a full-length foggy polypeptide or foggy polypeptide antagonist sequence as disclosed herein. Such foggy polypeptide variants may come in multiple forms. For example, “substitutional” variants are those that have at least one amino acid residue in a native sequence removed and a different amino acid inserted in its place at the same position. The substitutions may be single, where only one amino acid in the molecule has been substituted, or they may be multiple, where two or more amino acids have been substituted in the same molecule. “Insertional” variants are those with one or more amino acids inserted immediately adjacent to an amino acid at a particular position in a native sequence. Immediately adjacent to an amino acid means connected to either the α-carboxyl or α-amino functional group of the amino acid. “Deletional” variants are those with one or more amino acids in the native amino acid sequence removed. Ordinarily, deletional variants will have one or two amino acids deleted in a particular region of the molecule. Polypeptide variants also include covalent modifications to residues in addition to epitope-tagged heterogeneous foggy polypeptides and antagonists.

Alternatively, a foggy polypeptide variant or a foggy polypeptide antagonist variant will have at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a full-length native sequence foggy polypeptide or foggy polypeptide antagonist sequence as disclosed herein, or any other specifically defined fragment of a full-length foggy polypeptide sequence as disclosed herein. Ordinarily, foggy variant polypeptides or foggy variant polypeptide antagonists are at least about 100 amino acids in length, alternatively at least about 200 amino acids in length, alternatively at least about 300 amino acids in length, alternatively at least about 400 amino acids in length, alternatively at least about 500 amino acids in length, alternatively at least about 600 amino acids in length, alternatively at least about 700 amino acids in length, alternatively at least about 800 amino acids in length, alternatively at least about 850 amino acids in length, alternatively at least about 900 amino acids in length, alternatively at least about 950 amino acids in length, alternatively at least about 975 amino acids in length, alternatively at least about 1000 amino acids in length, alternatively at least about 1025 amino acids in length, alternatively at least about 1050 amino acids in length, alternatively at least about 1075 amino acids in length, alternatively at least about 1100 amino acids in length, or more.

“Percent (%) amino acid sequence identity” with respect to the foggy polypeptide or foggy polypeptide antagonist sequences identified herein is defined as the percentage of amino acid residues in a candidate sequence that are identical with the amino acid residues in the specific foggy polypeptide/foggy polypeptide antagonist sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, Tables 2 and 3 demonstrate how to calculate the % amino acid sequence identity of the amino acid sequence designated “Comparison Protein” to the amino acid sequence designated “PRO”, wherein “PRO” represents the amino acid sequence of a hypothetical PRO polypeptide of interest, “Comparison Protein” represents the amino acid sequence of a polypeptide against which the “PRO” polypeptide of interest is being compared, and “X, “Y” and “Z” each represent different hypothetical amino acid residues.

Unless specifically stated otherwise, all % amino acid sequence identity values used herein are obtained as described in the immediately preceding paragraph using the ALIGN-2 computer program. However, % amino acid sequence identity values may also be obtained as described below by using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix BLOSUM62. When WU-BLAST-2 is employed, a % amino acid sequence identity value is determined by dividing (a) the number of matching identical amino acid residues between the amino acid sequence of the foggy polypeptide or foggy polypeptide antagonist of interest having a sequence derived from the native foggy polypeptide or foggy polypeptide antagonist and the comparison amino acid sequence of interest (i.e., the sequence against which the foggy polypeptide of interest is being compared which may be a foggy variant polypeptide/antagonist therof) as determined by WU-BLAST-2 by (b) the total number of amino acid residues of the foggy polypeptide or foggy polypeptide variant of interest. For example, in the statement, “a polypeptide comprising an the amino acid sequence A which has or having at least 80% amino acid sequence identity to the amino acid sequence B,” the amino acid sequence A is the comparison amino acid sequence of interest and the amino acid sequence B is the amino acid sequence of the foggy polypeptide or foggy polypeptide antagonist of interest.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http:/www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value =0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“Foggy variant polynucleotide” or “foggy variant nucleic acid sequence” means a nucleic acid molecule which encodes an active foggy polypeptide as defined below and which has at least about 80% nucleic acid sequence identity with a nucleotide acid sequence encoding a full-length native sequence foggy polypeptide sequence or antagonist thereof as disclosed herein, or any other fragment of a full-length foggy polypeptide sequence or antagonist thereof as disclosed herein. Ordinarily, a foggy variant polynucleotide will have at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity with a nucleic acid sequence encoding a full-length native sequence foggy polypeptide or foggy polypeptide sequence antagonist sequence as disclosed herein, or any other fragment of a full-length foggy polypeptide of foggy polypeptide antagonist sequence as disclosed herein. Variants do not encompass the native nucleotide sequence.

“Percent (%) nucleic acid sequence identity” with respect to the foggy- or foggy antagonist-encoding nucleic acid sequences identified herein is defined as the percentage of nucleotides in a candidate sequence that are identical with the nucleotides in the foggy or foggy antagonist nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. For purposes herein, however, % nucleic acid sequence identity values are generated using the sequence comparison computer program ALIGN-2, wherein the complete source code for the ALIGN-2 program is provided in Table I below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5, demonstrate how to calculate the % nucleic acid sequence identity of the nucleic acid sequence designated “Comparison DNA” to the nucleic acid sequence designated “PRO-DNA”, wherein “PRO-DNA” represents a hypothetical PRO-encoding nucleic acid sequence of interest, “Comparison DNA” represents the nucleotide sequence of a nucleic acid molecule against which the “PRO-DNA” nucleic acid molecule of interest is being compared, and “N”, “L” and “V” each represent different hypothetical nucleotides.

Unless specifically stated otherwise, all % nucleic acid sequence identity values used herein are obtained as described in the immediately preceding paragraph using the ALIGN-2 computer program. However, % nucleic acid sequence identity values may also be obtained as described below by using the sequence comparison program NCBI-BLAST2 (Altschul et al., Nucleic Acids Res. 25:3389-3402 (1997)). The NCBI-BLAST2 sequence comparison program may be downloaded from http:/www.ncbi.nlm.nih.gov or otherwise obtained from the National Institute of Health, Bethesda, MD. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

In other embodiments, foggy variant or foggy variant antagonist polynucleotides are nucleic acid molecules that encode an active foggy polypeptide or foggy variant antagonist and which are capable of hybridizing, preferably under stringent hybridization and wash conditions, to nucleotide sequences encoding a full-length foggy polypeptide or foggy polypeptide antagonist as disclosed herein. Foggy variant polypeptides or antagonists thereof may be those that are encoded by a foggy variant polynucleotide or antagonists, respectively.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the foggy polypeptide natural environment will not be present. Ordinarily, however, isolated polypeptide will be prepared by at least one purification step.

An “isolated” foggy polypeptide-encoding nucleic acid or foggy polypeptide antagonist-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers, for example, single anti-foggy monoclonal antibodies (including agonist, antagonist, and neutralizing antibodies), anti-foggy antibody compositions with polyepitopic specificity, single chain anti-foggy antibodies, and fragments of anti-foggy antibodies (see below). The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.001 5 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5× SSC (0.75 M NaCl, 0.075 M sodium citrate phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a PRO polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

“Active” or “activity” for the purposes herein refers to form(s) of a foggy polypeptide which retain the ability to promote, cause or result in transcription elongation. In a specific aspect, the transcription elongation results in the formation of dopaminergic neurons. In a similar vein, “activity” of a foggy polypeptide antagonist refers to the ability to attenuate, disrupt or terminate transcription elongation, specifically, transcription elongation which results in the formation of serotonergic neurons.

The term “antagonist” is used in the broadest sense, and includes any molecule that partially or fully blocks, inhibits, or neutralizes a biological activity of a native foggy polypeptide disclosed herein. The term “foggy mutant” is used to describe an organism which does not express therapeutically effective amounts of foggy—and exhibits in vivo the effects of foggy antagonism. In a similar manner, the term “agonist” is used in the broadest sense and includes any molecule that mimics a biological activity of a native foggy polypeptide disclosed herein. Suitable agonist or antagonist molecules specifically include agonist or antagonist antibodies or antibody fragments, fragments or amino acid sequence variants of native foggy polypeptides, peptides, antisense oligonucleotides, small organic molecules, etc. Methods for identifying agonists or antagonists of a foggy polypeptide may comprise contacting a foggy polypeptide with a candidate agonist or antagonist molecule and measuring a detectable change in one or more biological activities normally associated with the foggy polypeptide.

“Treatment” refers to both therapeutic treatment and prophylactic or preventative measures, wherein the object is to prevent or slow down (lessen) the targeted pathologic condition or disorder. Accordingly, “treatment” refers to both therpeutic treatment and prophylactic of preventative measures. Those in need of treatment include those already with the disorder as well as those prone to have the disorder or those in whom the disorder is to be prevented. In the treatment of a disorder characterized by the degeneration of dopaminergic or serotonergic neurons, a therpeutic agent may directly decrease or increase the magnitude of the response of a pathological component of the disorder (e.g., diminished neural function), or render the disease more susceptible to threatment by other therapeutic agents.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of treatment refers to any animal classified as a mammal, including humans, domestic and farm animals, and zoo, sports, or pet animals, such as dogs, cattle, horses, sheep, pigs, goats, rabbits, cats, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., 1995, Protein Eng. 8(10):1057-1062); single-chain antibody molecule and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

The word “label” when used herein refers to a detectable compound or composition which is conjugated directly or indirectly to the antibody so as to generate a “labeled” antibody. The label may be detectable by itself (e.g. radioisotope labels or fluorescent labels) or, in the case of an enzymatic label, may catalyze chemical alteration of a substrate compound or composition which is detectable.

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a foggy polypeptide or antagonist) to a mammal. The components of the liposome are commonly arranged in a bilayer formation, similar to the lipid arrangement of biological membranes.

A “small molecule” is defined herein to have a molecular weight below about 500 Daltons.

A “disorder” is any condition that would benefit from treatment with or from application of a molecule which results in the formation of dopaminergic or serotonergic neurons. Examples of disorders that would benefit from implantation of or the formation of dopaminergic neurons are those associated with improper postural reflexes, movement and reward-associated behaviors, including, Parkinson's disease, schizophrenia and drug addiction. Examples of disorders that would benefit from implantation of or the formation of serotonergic neurons are those characterized by abnormalities in awareness, arousal, behavior, and food intake, including aggression, depression (including suicidal behavior), schizophrenia and anorexia/bulimia.

An “effective amount” of foggy polypeptide is at least the minimum amount that is sufficient to promote, cause or result in transcription elongation, e.g., such as that which results in the formation of serotonergic neurons from neuroprogenitor cells. Similarly, an “effective amount” of a foggy polypeptide antagonist is at least the minimum amount that is sufficient to attenuate, disrupt or terminate transcription elongation, e.g., such as that which results in the formation of dopaminergic neurons from neuroprogenitor cells. An “effective amount” of neuronal survival factor is such amount so as to promote the survival of a greater population of neuronal cells that would otherwise exist without the survival factor.

A “neuronal survival factor” is any substance which causes neurons (either in cell culture or as a transplanted mass) to which the factor is placed into contact with, to survive for period of time greater than would otherwise occur. For example, U.S. Pat. No. 5,733,875 describes a method of using Glial-derived neurotrophic factor (GDNF) to protect or prevent epileptic seizures. GDNF is a known agent having trophic activity for embryonic midbrain ventral mesencephalic dopaminergic neurons in vitro. Lin et al., 1993, Science 260: 1130-1132; Lin et al., 1994, J. Neurochem. 63: 758-768. Recombinant human GDNF has also been demonstrated to induce sprouting of dopaminergic fibers in vivo (Hudson et al., 1993, Soc. Neurosci. Abstr. 19: 652), increase dopamine turnover in the substantia nigra of rats (Hudson et al., supra, Miller et al., 1994, Soc. Neurosci. Abstr. 20: 535-7), protect neurons against 6-OHDA lesions, and augment growth and fiber formation of rat fetal transplants of nigral tissue in oculo, Stromberg et al., 1993, Exp. Neurol. 124: 401-412.

Brain-derived neurotrophic factor (BDNF) is a trophic factor for peripheral sensory neurons, dopaminergic neurons and retinal ganglia. Henderson et al., 1993, Restor. Neurol. Neurosci. 5: 15-28. BDNF has also been shown to prevent normally-occurring cell death both in vitro and in vivo. Hofer and Barde, 1988, Nature 331: 161-262. Neurotrophin-3 is found both in the central and peripheral nervous systems and is capable of promoting the survival of sensory and sympathetic neurons, including dorsal root ganglia (DRG) explants. Ciliary NeuroTrophic Factor (CTNF) promotes the survival of chicken embryo ciliary ganglia in vitro and was also found to support survival of cultured sympathetic, sensory and spinal motor neurons. Ip et al., 1991, J. Physiol. 85: 123-130. Local administration of this protein to the lesion site of newborn rates had been shown to prevent the degeneration of the corresponding motor neurons. CNTF also rescued motor neurons from development cell death. Henderson et al., 1993, Restor. Neurol. Neurosci. 5: 15-28.

Additional neuronal survival factors include nerve growth factors (NGF), aGF, neurotrophin-3 (NT-3), neurotrophin-4 (NT-4), aFGF, IL-1b, TNFa-, insulin-like growth factor (IGF-1, IGF-2), transforming growth factor beta (TGF-β, TGF-β1).

A “therapeutically effective amount” of progenitor cells is such amount which arrests or ameliorates the physiological effects caused by the loss or damage to dopaminergic or serotonergic neurons. A suitable range of cells can range from about 100 to about 500,000 active neurons. Preferably, the range is about 500 to about 500,000, most preferably about 1,000 to about 500,000. A “therapeutically effective amount” of foggy polypeptide is at least the minimum amount that is sufficient to attenuate, alleviate or otherwise improve the condition of a patient afflicted with a disorder characterized by a deficiency of serotonergic neurons. A “therapeutically effective amount” of foggy polypeptide is at least the minimum amount that is sufficient to attenuate, alleviate or otherwise improve the condition of a patient afflicted with a disorder characterized by a deficiency of dopaminergic neurons.

“Dopaminergic (DA) neurons” refers to neurons which secrete the neurotransmitter dopamine. They innervate the striatum, limbic system, and neocortex and reside in the ventral midbrain together with several other classes of neurons including motoneurons. DA neurons control postural reflexes, movement and reward-associated behaviors. The loss of DA neurons results in Parkinson's disease and their abnormal function have been associated with schizophrenia and drug addiction.

“Serotonergic (5HT) neurons” refers to neurons which secrete the neurotransmitter serotonin (5-hydroxytryptamine). 5HT neurons typically have a slow, rhythmic pattern of firing and are concentrated in the ventral and ventrolateral aspects of the hindbrain and innervate most parts of the central nervous system including the cerebral cortex, limbic system and spinal cord. 5HT neurons control levels of awareness, arousal, behavior and food intake. The abnormal function of serotonergic neurons has been linked to aggression, depression (including suicidal behavior) and schizophrenia.

“Neuroprogenitor cells” are cells which give rise to or differentiate into neurons. They have been observed to differentiate into various neuronal classes dependent on their relative placement along the anterior-posterior and dorsal-ventral axis. Furthermore, neuroprogenitor cells for use with the present invention will differentiate into serotongeric neurons when contacted with an effective amount of a foggy polypeptide, and will differentiate into dopaminergic neurons when contacted with an effective amount of a foggy polypeptide antagonist.

TABLE 4

| | | |
|---|---|---|
| PRO-DNA | NNNNNNNNNNNNNN | (Length = 14 nucleotides) |
| Comparison | NNNNNNLLLLLLLLLL | (Length = 16 nucleotides) |
| DNA | | |

% nucleic acid sequence identity = (the number of identically matching nucleotides between the two nucleic acid sequences as determined by ALIGN-2) divided by (the total number of nucleotides of the PRO-DNA nucleic acid sequence) = 6 divided by 14 = 42.9%

TABLE 5

| | | |
|---|---|---|
| PRO-DNA | NNNNNNNNNNNN | (Length = 12 nucleotides) |
| Comparison DNA | NNNNLLLVV | (Length = 9 nucleotides) |

% nucleic acid sequence identity = (the number of identically matching nucleotides between the two nucleic acid sequences as determined by ALIGN-2) divided by (the total number of nucleotides of the PRO-DNA nucleic acid sequence) = 4 divided by 12 = 33.3%

II. Compositions and Methods of the Invention

A. Foggy is the key Differentiation Marker for Neuronal Development.

The present invention describes and characterizes the nucleotide and amino acid sequence of foggy, a polypeptide corresponding to a zebrafish mutant whose phenotype was originally identified in a genetic screen for mutations affecting the expression of tyrosine hydroxylase (TH). Foggy has now been identified as the key differentiation marker for dopaminergic (DA) and noradrenergic (NA) neurons. Guo et al., 1999, Devel. Biol. 208: 473-87. The foggy mutant embryos appear morphologically normal but show deficits in hypothalamic and retinal dopaminergic (DA) neurons, hindbrain noradrenergic (NA) neurons and the neural crest-derived sympathetic NA neurons. Surprisingly, the deficits in hypothalamic DA neurons were accompanied by an increase in the number of neighboring serotonergic (5HT) neurons. Neural development was also affected in the developing retina, where deficits in amacrine and photoreceptor neurons were observed. In contrast, the retinal ganglion neurons were not defective, and even exhibited a small increase in numbers. Many other cases of neurons including 5HT and Mauthner neurons in the hindbrain, GABAergic inter and motor neurons in the spinal cord, neural crest-derived dorsal root ganglia and the NA arch-associated CA (AAC) cells, developed normally in the foggy mutant. Positional cloning disclosed that the molecular cause of the mutant phenotype is a point mutation in the nuclear protein that is structurally related to the yeast and human Spt5. Biochemical analysis of foggy revealed that it is dual regulator of mRNA transcription elongation and that the mutation specifically disrupts its inhibitory but not the stimulatory activity. Taken together, these findings provide molecular, genetic and biochemical evidence that transcription elongation is indeed a key regulatory point for gene expression in vertebrates, and that precise control of transcription elongation, together with transcription initiation, is essential for proper neural development. Applicants describe herein, a zebrafish mutant that selectively affects the development and identity of multiple neuronal types in the central (CNS) and peripheral (PNS) nervous systems. Further molecular, genetic and biochemical analyses demonstrate that the mutant phenotype is caused by a mutation that disrupts the inhibitory but not stimulatory function of zSpt5 in transcription elongation. These findings demonstrate that regulation of transcription elongation constitutes an essential physiological control step in gene expression during neural development.

1. Transcription Elongation as a Regulated Process

The mutant foggy phenotype highlights the significance of transcription elongation as a regulated process in vivo. This type of regulation affords a level of control which is not available to the transcription initiation apparatus. First, it permits for a very rapid response to changes in the environment or to extracellular stimuli. For example, Drosophila may be able to survive heat since its heat shock protein hsp70 transcript is regulated by anti-termination mechanism which is removed upon heat stimulus; thus, the transcript is rapidly completed and is available for protein synthesis. Also, regulation of transcription elongation permits ongoing control of gene transcription. For example, the human dystrophin gene spans more than 2000 kb and would take more than 50 hours to fully transcribe. Uptain et al., supra. Without intervention at the transcription elongation stage, once initiated, transcription of such a gene will be without regulation for over 2 days. Conversely, having a pool of nearly complete mRNA with RNA Pol II arrested or posing near the end of the dystorphin transcripts, would allow, in response to anti-termination signal, for rapid production of this protein. Finally, combinatorial usage of transcription initiation and elongation factors would allow control of spatial and temporal gene expression with a relatively small number of factors. Flexibility, rapid response and sophisticated control on gene expression are particularly important during development of the nervous system. As a result, progenitors have to constantly monitor and respond to changes in the extra-cellular environment and achieve large cellular diversity in a span of a few hours. Anderson and Jan, 1997; Ericson et al., 1996, Cell 87: 661-73.

2. Specificity of the Foggy Mutant Phenotype

Applicants describe a point mutation herein which disrupts the inhibitory but not the stimulatory activity of Spt5 in transcription elongation, resulting in the failure of specific sub-classes of neurons and cells to complete their development. The mutant phenotype exerts remarkable spatial specificity, spanning both the CNS and PNS and influencing multiple classes of neurons. For example, within the neural crest-derived lineage are found deficits in iridophores, melanocytes, and sympathetic neurons but not in the dorsal root ganglia neurons. In the CNS, there are deficits in DA and NA, but not in 5HT or Mauthner neurons. The deficits do not simply affect the production of a single type of neurotransmitters as dopamine, noradrenalin and acetyl choline producing neurons are all affected. Also, the influence on a given neurotransmitter appear to be region specific as illustrated by the fact that NA producing cells are absent from the hindbrain (LC neurons) but are normal adjacent to the heart (AAC cells), whereas 5HT+ neurons are increased in the hypothalamic but not the hindbrain.

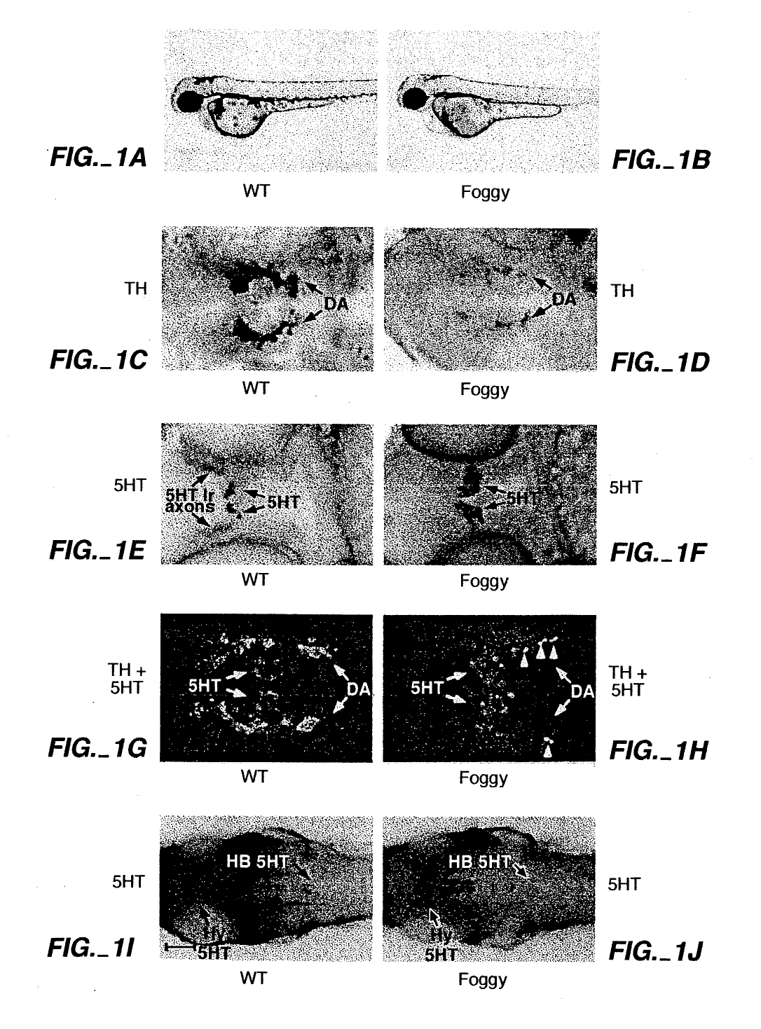

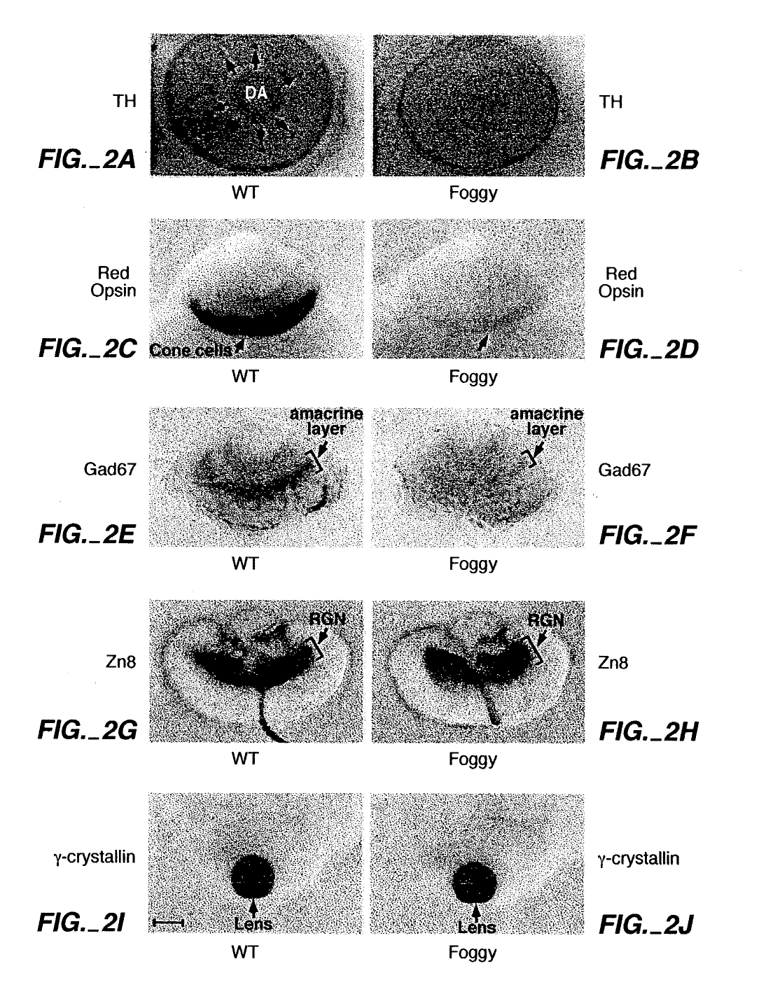

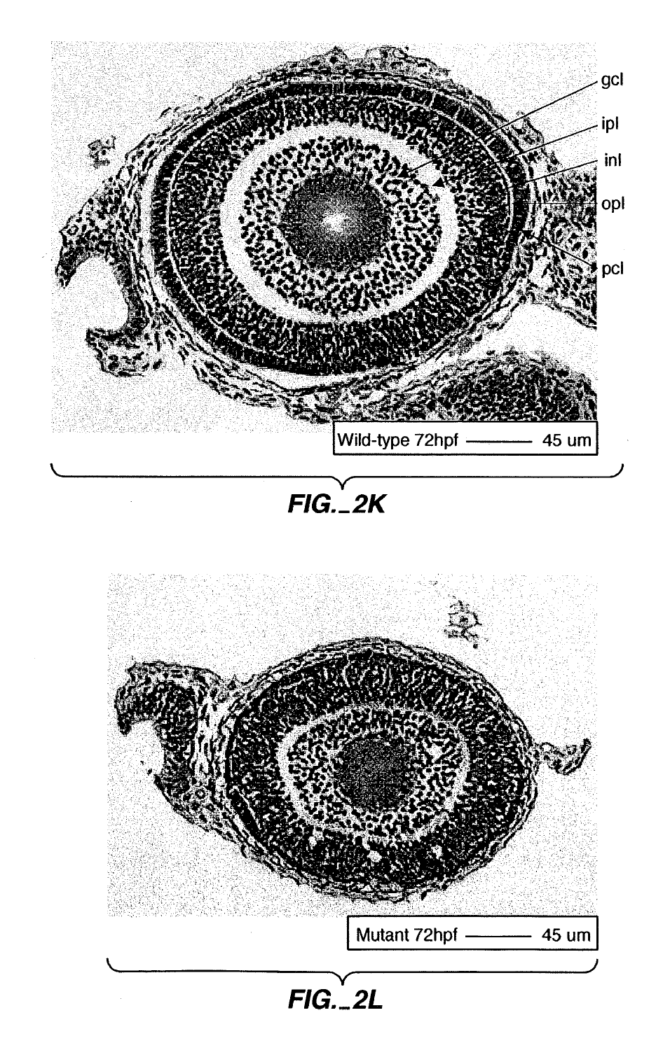

Temporally, the mutation appears to influence later stages of differentiation and perhaps subprograms of neuronal identity. This conclusion is supported by several findings. First, expression of Pan-neuronal marker tubulin appears normal in the 48 hpf embryo. Guo et al., supra. Second, the homeobox containing gene phox2a, which controls NA identity in LC and sympathetic neurons (Guo et al., 1999, Neuron 24: 555-66; Morin et al., 1997, Neuron 18: 411-423), is normally expressed in the progenitors for these cells but subsequently disappears before these cells express any neurotransmitter synthesis enzyme. Finally, in the hypothalamus, a surplus of 5HT neurons is observed apparently at the expense of DA neurons. The notion that foggy affects the final neurotransmitter identity is further supported by the fact that the neurons that remain in foggy fail to complete their differentiation. For example, the few DA neurons that are present in the foggy hypothalamus express lower levels of TH (FIGS. 1B&C), whereas the left over amacrine neurons appear to express low levels of the GABA synthesis enzyme GAD (FIGS. 2E & F).

In the retina, deficits of multiple neurons were observed and an apparent small increase of ganglion neurons. The fact that in the retina, ganglion neurons are the first to appear raise the possibility that in the absence of foggy, neural progenitors undergo premature differentiation and are no longer available to assume latter cell fates. In the hypothalamus, DA neurons, which suffer deficits and SHT neurons, which are present in excess, seem to develop simultaneously (data not shown). However, in the chick mid/hindbrain region, development of 5HT neurons appear to precede that of the DNA counterparts (unpublished data) suggesting that small temporal differences in the developmental period between DNA and 5HT may exist also in the fish.

3. Spatial and Temporal Regulation of Transcription Elongation by Foggy

Foggy is widely expressed during development and appears to be ubiquitously expressed in the adult. Yamaguchi et al., supra. Moreover, the drug DRB, whose action is dependent on DSIP, inhibits mRNA synthesis of over 95% of the cellular Pol II transcripts (Sehgal et al., 1976, Cell 9: 473-80), suggesting that Spt4 and Spt5 are involved in regulating the transcription of most, if not all, of the class II genes. Despite that, the foggy mutation results in selective developmental changes and appears to affect only a subset of genes. The phenomenon of a mutation in a widely expressed protein leading to cell type-specific cell fate changes or deficits is often observed but not fully understood. For example, disruption of the orphan steroid receptors Nurr I, which is widely expressed in the CNS, lead to restricted deficits in midbrain DA neurons in the mouse. Saucedo-Cardenas et al., 1998, Proc. Natl. Acad. Sci. USA 95: 4013-4018; Zetterstrom et al., 1997, Science 276: 248-250. Likewise, mutation in ubiquitous, EGF-related molecule, one-eyed pinhead, causes deficits in the prechordal plate but not notochord, even though during gastrulation it is expressed in both of these tissues. Zhang et al., 1998, Cell 92: 241-51.

At least three possible explanations exist for the tissue specific phenotype of foggy. First, it is possible that the foggy mutation is partially compensated for by putative foggy/zSpt5 family members, or by maternal contribution of foggy mRNA. Alternatively, although widely expressed, foggy/zSpt5 may physiologically affect only a subset of genes, for which elongation constitutes a rate-limiting step. Consistent with this possibility is the finding that mutations in Pol II that decrease the elongation rate cause homeotic transformation in Drosophila. Chen et al., 1993; Mol. Cell. Biol. 13: 4214-22; Coulter and Greenleaf, 1985, J. Biol. Chem. 260: 13190-8. Finally it is possible that the activity or nuclear localization rather than expression of foggy/zSpt5 could be regulated by other factors in a tissue or temporal specific manner or in response to extra-cellular signals. Since the mutation affects the inhibitory function of foggy/zSpt5, it would be apparent that the observed phenotype is caused by aberrant expression of genes, the transcription of which is normally restricted or silenced by premature termination. Overexpression of such genes in a wrong cell type or during inappropriate developmental stages could result in neuronal deficits. The specificity of the foggy phenotype argues that the affected genes are likely to encode regulatory rather than house keeping proteins. Better understanding of the underlying mechanisms that unifies that observed developmental aberrations would require the identification of foggy/zSpt5 target genes.

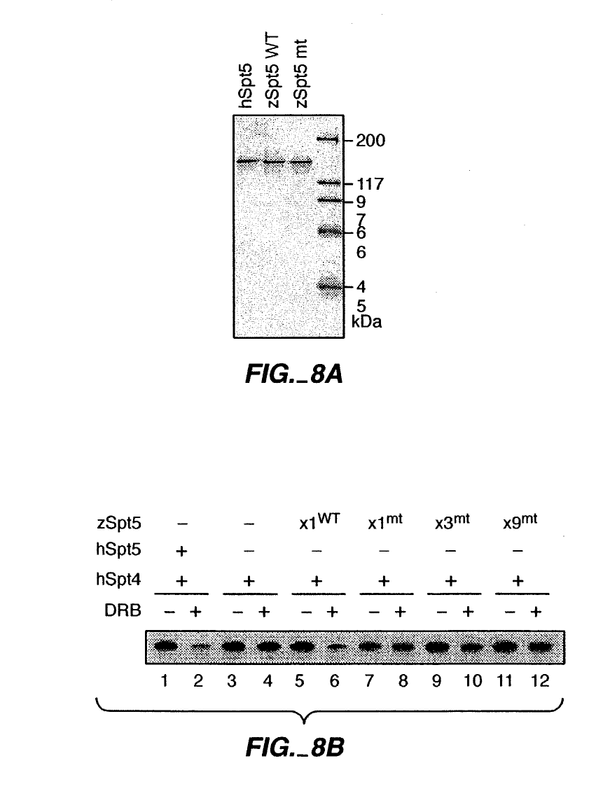

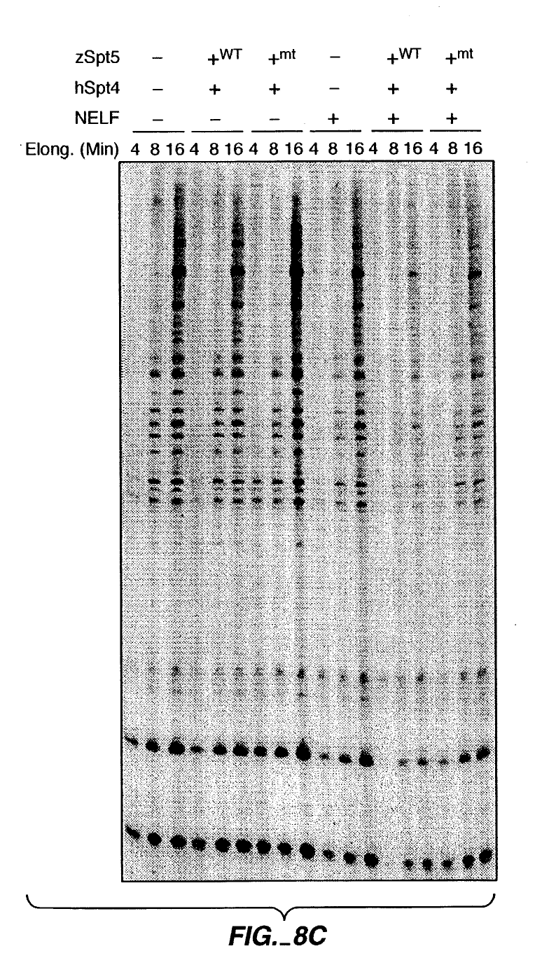

Even though foggy has the ability to influence transcription of all Pol II genes, several lines of evidence suggest that it normally plays a regulatory role in gene expression. First, foggy/zSpt5 as well as its mammalian homolog are not required for transcription elongation in vitro. (FIG. 8). Second, foggy/zSpt5 appears to have dual affects on transcription elongation and can act as a negative or positive regulator under different environmental conditions (presence of DRB or low nucleotide concentrations, respectively, FIG. 8). Third, the fact that the foggy/zSpt5 mutant lost its inhibitory activity argues that the phenotype is not a result of a basic inability of Pol II to extend RNA transcripts.

Spt5 was shown to bind Spt4, the large subunit of Pol II and the NELF protein complex and through this interaction to inhibit transcription elongation by RNA Pol II. Although Spt5 does not bind the carboxy Terminal Domain (CTD) of Pol II, its inhibitory activity is counteracted by phosphorylation of the CTD by DRB-sensitive kinase P-TEFb.

Structure function analysis of hSpt5 (Yamaguchi et al., supra) revealed that amino acids 176-270 are responsible for binding to Spt4 whereas amino acids 313-430 are required for binding to Pol II. The function of the C-terminal domain of Spt5 had not been previously determined. The present application exemplifies that a single amino acid substitution of zSpt5 at position 1012 (corresponding to amino acid 1014 of hSpt5) inactivates the negative function of DSIF. A possible function of the C-terminal domain of Spt5 is as a binding domain for NELF complex which is critically required for the inhibitory activity of Spt5. Yamaguchi et al., supra. Alternatively, the C-terminal domain may bind a yet unidentified modulator of transcription elongation. The mechanism by which the Spt5 inhibits elongation is not well understood. One possibility is that, the DSIF/NELF complex may function like the bacterial Nus A/Nun complex. In bacteria, the Nus A protein was shown to activate the transcription termination through the coliphage HK022 elongation inhibitor Nun, which, in the presence of NuseA binds both DNA and RNA and anchors the nascent transcript to its double stranded DNA template. Watnick and Gottesman, 1999, Science 286: 2337-2339. Spt5, which contains 4 Nus homology domains and the NELF complex—the latter of which contains and RNA binding component may each act by similar mechanisms. Yamaguchi et al., supra. Alternatively, Spt5 in conjuction with Spt4, NELF, the transcription elongation and chromatin binding protein-Spt6 and additional members of the Spt proteins influence the compactness and organization of histones and prevents the movement of Pol II through the nucleosomes. Bortvin and Winston, 1996, Science 272: 1473-6; Compagnone-Post and Osley, 1996, Genetics 143: 1543-54; Swanson and Winston, supra. In addition to its inhibitory activity, Spt5 acts as a stimulator of transcription elongation at low nucleotide concentration. Surprisingly, this stimulatory activity of Spt5 is retained by the mutant. These findings support the idea that the inhibitory and stimulatory activities of Spt5 reside in distinct domains or that the stimulatory function of Spt5 may be independent of NELF.

4. Regulation of Foggy by Extrinsic Signals

Whereas there is well documented evidence for control of transcription initiation by a multitude of signal transduction pathways and extracellular stimuli, to date the only reported link between transcription elongation and extrinsic factors in multi-cellular organisms stem from studies on Notch signaling in C. elegans. The Notch signaling system is involved in multiple, often binary, cell fate choices including the decision whether to become neuronal or epidermal cell, whether to develop as ganglion cells, cone or rod photoreceptor and whether to assume a given neural crest-derived cell fate. The prevailing model for signal transduction by Notch involves direct interaction between its cytoplasmic domain that is cleaved following ligand binding, and homologs of the transcription initiation factor suppressor of Hairless (Su(H)). However, in addition to Su(H), Notch appears to mediate signaling through other activators or suppressors of transcription initiation such as NF-κB, homologs of Groucho and Mastermind (Artavanis-Tsakonas et al., supra; Lewis, 1998, Sem. Cell Devel. Biol. 9: 583-9; Milner and Bigas, 1999, Blood 93: 2431-2448) as well as through yet unidentified transcription factors. Shawber et al., 1996, Devel. 122: 3765-3773; Wang et al., 1997, Devel. 124: 4435-46.

Interestingly, a yeast two-Hybrid screen identified components that act downstream of Notch in C. elegans revealed that Notch physically interacts with EMB-5, the worm homolog of Spt6. Moreover, genetic studies established that mutations in EMB/Spt6 affect the penetrance of constitutively active or mutated Notch on cell fate decision. Hubbard et al., 1996, Science 273: 112-5. Taken together with the report that Spt6 and Spt5 genetically and physically interact (Swanson and Winston, 1992, supra), these findings raise the possibility that Spt5 activity may be a downstream mediator of the Notch pathway. Alternatively, since mammal members of the FGF protein family were shown to induce 5HT neurons at the expense of DA neurons in the midbrain (Ye et al., 1998, Cell 93: 755-66), it is possible that foggy/zSpt5 is linked to the FGF signaling system.

In summary the present application provides evidence that negative regulation of transcription elongation is essential for normal development in multi-cellular organisms. The findings further support the hypothesis that positive and negative transcription elongation factors may have a role similar to those of transcription initiation factors in the control of temporal and spatial gene expression during neural development.

B. Foggy Polypeptide Variants

In addition to the full-length native sequence foggy polypeptides described herein, it is contemplated that foggy variants can be prepared. Foggy variants can be prepared by introducing appropriate nucleotide changes into the foggy-encoding DNA, and/or by synthesis of the desired foggy polypeptide. Those skilled in the art will appreciate that amino acid changes may alter the ability of foggy to modulate transcription elongation.

Variations in the native full-length sequence foggy or in various domains of the foggy polypeptide described herein, can be made, for example, using any of the techniques and guidelines for conservative and non-conservative mutations set forth, for instance, in U.S. Pat. No. 5,364,934. Variations may be a substitution, deletion or insertion of one or more codons encoding foggy that results in a change in the amino acid sequence of foggy as compared with the native sequence foggy. Optionally the variation is by substitution of at least one amino acid with any other amino acid in one or more of the domains of foggy. Guidance in determining which amino acid residue may be inserted, substituted or deleted without adversely affecting the desired activity may be found by comparing the sequence of foggy with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

Foggy polypeptide fragments are provided herein. Such fragments may be truncated at the N-terminus or C-terminus, or may lack internal residues, for example, when compared with a full length native protein. Certain fragments lack amino acid residues that are not essential for a desired biological activity of the foggy polypeptide.

Foggy fragments may be prepared by any of a number of conventional techniques. Desired peptide fragments may be chemically synthesized. An alternative approach involves generating foggy fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, foggy polypeptide fragments share at least one biological and/or immunological activity with the native foggy polypeptide disclosed herein.

In particular embodiments, conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are introduced and the products screened.

TABLE 6

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of the PRO polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gin, his, lys, arg;

(5) residues that influence chain orientation: gly, pro; and

(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., 1986, Nucl. Acids Res., 13:4331; Zoller et al., 1987, Nucl. Acids Res., 10:6487], cassette mutagenesis [Wells et al., 1985, Gene, 34:315], restriction selection mutagenesis [Wells et al., 1986, Philos. Trans. R. Soc. London SerA, 317:415] or other known techniques can be performed on the cloned DNA to produce the PRO variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, 1989, Science, 244: 1081-1085 (1989)]. Alanine is also typically preferred because it is the most common amino acid. Further, it is frequently found in both buried and exposed positions [Creighton, The Proteins, (W. H. Freeman & Co., N.Y.); Chothia, 1976, J. Mol. Biol., 150:1 (1976)]. If alanine substitution does not yield adequate amounts of variant, an isoteric amino acid can be used.

C. Modifications of Foggy

Covalent modifications of foggy are included within the scope of this invention. One type of covalent modification includes reacting targeted amino acid residues of a foggy polypeptide with an organic derivatizing agent that is capable of reacting with selected side chains or the N- or C-terminal residues of foggy. Derivatization with bifunctional agents is useful, for instance, for crosslinking foggy to a water-insoluble support matrix or surface for use in the method for purifying anti-foggy antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Addition of glycosylation sites to the foggy polypeptide may be accomplished by altering the amino acid sequence. The alteration may be made, for example, by the addition of, or substitution by, one or more serine or threonine residues to the native sequence foggy (for O-linked glycosylation sites). The PRO amino acid sequence may optionally be altered through changes at the DNA level, particularly by mutating the DNA encoding the PRO polypeptide at preselected bases such that codons are generated that will translate into the desired amino acids.

Another means of increasing the number of carbohydrate moieties on the PRO polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the foggy polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., 1987, Arch. Biochem. Biophys., 259:52 and by Edge et al., Anal. Biochem., 118:131 (1981). Enzymatic cleavage of carbohydrate moieties on polypeptides can be achieved by the use of a variety of endo- and exo-glycosidases as described by Thotakura et al., 1987, Meth. Enzymol., 138:350.

Another type of covalent modification of foggy comprises linking the foggy polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

Foggy polypeptides of the present invention may also be modified in a way to form a chimeric molecule comprising foggy fused to another, heterologous polypeptide or amino acid sequence.

In one embodiment, such a chimeric molecule comprises a fusion of the foggy with a tag polypeptide which provides an epitope to which an anti-tag antibody can selectively bind. The epitope tag is generally placed at the amino- or carboxyl-terminus of the foggy polypeptide. The presence of such epitope-tagged forms of the foggy can be detected using an antibody against the tag polypeptide. Also, provision of the epitope tag enables foggy to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., 1988, Mol. Cell. Biol., 8:2159-2165); the c-myc tag and the 8F9, 3C7, 6E10, G4, B7 and 9E10 antibodies thereto (Evan et al., 1985, Molecular and Cellular Biology, 5:3610-3616); and the Herpes Simplex virus glycoprotein D (gD) tag and its antibody (Paborsky et al., 1990, Protein Engineering, 3(6):547-553). Other tag polypeptides include the Flag-peptide (Hopp et al., 1988, BioTechnology, 6:1204-1210); the KT3 epitope peptide (Martin et al., 1992, Science, 255:192-194); an α-tubulin epitope peptide (Skinner et al., 1991, J. Biol. Chem., 266:15163-15166); and the T7 gene 10 protein peptide tag (Lutz-Freyermuth et al, 1990, Proc. Natl. Acad. Sci. USA, 87:6393-6397).

In an alternative embodiment, the chimeric molecule may comprise a fusion of the foggy with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a foggy polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

D. Preparation of Foggy

The description below relates primarily to production of foggy by culturing cells transformed or transfected with a vector containing foggy nucleic acid. It is, of course, contemplated that alternative methods, which are well known in the art, may be employed to prepare foggy. For instance, the foggy sequence, or portions thereof, may be produced by direct peptide synthesis using solid-phase techniques [see, e.g., Stewart et al., Solid-Phase Peptide Synthesis, W. H. Freeman Co., San Francisco, Calif. (1969); Merrifield, 1963, J. Am. Chem. Soc., 85:2149-2154. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the foggy may be chemically synthesized separately and combined using chemical or enzymatic methods to produce the full-length foggy.

1. Isolation of DNA Encoding Foggy

DNA encoding foggy may be obtained from a cDNA library prepared from tissue believed to possess the foggy mRNA and to express it at a detectable level. Accordingly, human foggy DNA can be conveniently obtained from a cDNA library prepared from human tissue, such as described in the Examples. The foggy-encoding gene may also be obtained from a genomic library or by known synthetic procedures (e.g., automated nucleic acid synthesis).

Libraries can be screened with probes (such as antibodies to the foggy or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding PRO is to use PCR methodology [Sambrook et al., supra; Dieffenbach et al., PCR Primer: A Laboratory Manual (Cold Spring Harbor Laboratory Press, 1995)].

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for PRO production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772(ATCC 53,635). Other suitable prokaryotic host cells include Enterobacteriaceac such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA ; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac)169 degP ompT rbs7 ilvG kan^{r}; E. coli W3 110 strain 40B4, which is strain 37D6 kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for PRO-encoding vectors. Saccharomyces cerevisiae is a commonly used lower eukaryotic host microorganism. Others include Schizosaccharomyces pombe (Beach and Nurse, 1981, Nature, 290: 140); EP 139,383 published 2 May 1985); Kluyveromyces hosts (U.S. Pat. No. 4,943,529; Fleer et al., 1991, Bio/Technology, 9:968-975 (1991)) such as, e.g., K. lactis (MW98-8C, CBS683, CBS4574; Louvencourt et al. J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated PRO are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., 1977, J. Gen Virol., 36:59); Chinese hamster ovary cells/-DHFR (CHO, Urlaub and Chasin, 1980, Proc. Natl. Acad. Sci. USA, 77:4216 (1980)); mouse sertoli cells (TM4, Mather, 1980, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

3. Selection and Use of a Replicable Vector

The nucleic acid (e.g., cDNA or genomic DNA) encoding foggy may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The foggy may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the foggy-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces a-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the foggy-encoding nucleic acid, such as DHFR or thymidine kinase. An appropriate host cell when wild-type DHFR is employed is the CHO cell line deficient in DHFR activity, prepared and propagated as described by Urlaub et al., 1980, Proc. Natl. Acad. Sci. USA, 77:4216. A suitable selection gene for use in yeast is the trp1 gene present in the yeast plasmid YRp7 (Stinchcomb et al., 1979, Nature, 282:39); Kingsman et al., 1979, Gene 7:141; Tschemper et al., 1980, Gene, 10:157). The trp1 gene provides a selection marker for a mutant strain of yeast lacking the ability to grow in tryptophan, for example, ATCC No. 44076 or PEP4-1 (Jones, 1977, Genetics 85:12).

Expression and cloning vectors usually contain a promoter operably linked to the foggy-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding foggy.

Examples of suitable promoting sequences for use with yeast hosts include the promoters for 3-phosphoglycerate kinase (Hitzeman et al., 1980, J. Biol. Chem., 255:2073) or other glycolytic enzymes (Hess et al., 1968, J. Adv. Enzyme Reg., 7:149); Holland, 1978, Biochemistry 17:4900), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Foggy transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the foggy by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the foggy coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding foggy.

Still other methods, vectors, and host cells suitable for adaptation to the synthesis of foggy in recombinant vertebrate cell culture are described in Gething et al., Nature, 293:620-625 (1981); Mantei et al., Nature, 281:40-46 (1979); EP 117,060; and EP 117,058.

4. Detecting Gene Amplification/Expression

Gene amplification and/or expression may be measured in a sample directly, for example, by conventional Southern blotting, Northern blotting to quantitate the transcription of mRNA (Thomas, Proc. Natl. Acad. Sci. USA, 1980, 77:5201-5205), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence foggy polypeptide or against a synthetic peptide based on the DNA sequences provided herein or against exogenous sequence fused to foggy DNA and encoding a specific antibody epitope.

5. Purification of Polypeptide

Forms of foggy may be recovered from culture medium or from host cell lysates. If membrane-bound, it can be released from the membrane using a suitable detergent solution (e.g. Triton-X® 100) or by enzymatic cleavage. Cells employed in expression of foggy can be disrupted by various physical or chemical means, such as freeze-thaw cycling, sonication, mechanical disruption, or cell lysing agents.

It may be desired to purify foggy from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the PRO. Various methods of protein purification may be employed and such methods are known in the art and described for example in Deutscher, 1990, Methods in Enzymology, 182; Scopes, Protein Purification: Principles and Practice, Springer-Verlag, New York (1982). The purification step(s) selected will depend, for example, on the nature of the production process used and the particular foggy produced.

E. General Uses for Foggy Polypeptides

Nucleotide sequences (or their complement) encoding foggy have various applications in the art of molecular biology, including uses as hybridization probes, in chromosome and gene mapping and in the generation of anti-sense RNA and DNA. Foggy nucleic acid will also be useful for the preparation of foggy polypeptides by the recombinant techniques described herein.

The full-length native sequence foggy gene, or portions thereof, may be used as hybridization probes for a cDNA library to isolate the full-length foggy CDNA or to isolate still other cDNAs (for instance, those encoding naturally-occurring variants of foggy or foggy from other species) which have a desired sequence identity to the native foggy sequence disclosed herein. Optionally, the length of the probes will be about 20 to about 50 bases. The hybridization probes may be derived from at least partially novel regions of the full length native nucleotide sequence wherein those regions may be determined without undue experimentation or from genomic sequences including promoters, enhancer elements and introns of native sequence foggy. By way of example, a screening method will comprise isolating the coding region of the foggy gene using the known DNA sequence to synthesize a selected probe of about 40 bases. Hybridization probes may be labeled by a variety of labels, including radionucleotides such as ³²p or ³⁵S, or enzymatic labels such as alkaline phosphatase coupled to the probe via avidin/biotin coupling systems. Labeled probes having a sequence complementary to that of the foggy gene of the present invention can be used to screen libraries of human cDNA, genomic DNA or mRNA to determine which members of such libraries the probe hybridizes to. Hybridization techniques are described in further detail in the Examples below.

Any EST sequences disclosed in the present application may similarly be employed as probes, using the methods disclosed herein.

Other useful fragments of the foggy nucleic acids include antisense or sense oligonucleotides comprising a singe-stranded nucleic acid sequence (either RNA or DNA) capable of binding to target foggy mRNA (sense) or foggy DNA (antisense) sequences. Antisense or sense oligonucleotides, according to the present invention, comprise a fragment of the coding region of foggy DNA. Such a fragment generally comprises at least about 14 nucleotides, preferably from about 14 to 30 nucleotides. The ability to derive an antisense or a sense oligonucleotide, based upon a cDNA sequence encoding a given protein is described in, for example, Stein and Cohen, 1988, Cancer Res. 48:2659) and van der Krol et al., 1988, BioTechniques 6:958, 1988).

Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. The antisense oligonucleotides thus may be used to block expression of foggy proteins. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e.,capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, CaPO₄-mediated DNA transfection, electroporation, or by using gene transfer vectors such as Epstein-Barr virus. In a preferred procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MuLV, N2 (a retrovirus derived from M-MuLV), or the double copy vectors designated DCT5A, DCT5B and DCT5C (see WO 90/13641).

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

Antisense or sense RNA or DNA molecules are generally at least about 5 bases in length, about 10 bases in length, about 15 bases in length, about 20 bases in length, about 25 bases in length, about 30 bases in length, about 35 bases in length, about 40 bases in length, about 45 bases in length, about 50 bases in length, about 55 bases in length, about 60 bases in length, about 65 bases in length, about 70 bases in length, about 75 bases in length, about 80 bases in length, about 85 bases in length, about 90 bases in length, about 95 bases in length, about 100 bases in length, or more.

The probes may also be employed in PCR techniques to generate a pool of sequences for identification of closely related foggy coding sequences.

Nucleotide sequences encoding a foggy can also be used to construct hybridization probes for mapping the gene which encodes foggy and for the genetic analysis of individuals with genetic disorders. The nucleotide sequences provided herein may be mapped to a chromosome and specific regions of a chromosome using known techniques, such as in situ hybridization, linkage analysis against known chromosomal markers, and hybridization screening with libraries.

Nucleic acids which encode foggy or its modified forms (e.g., such as antagonists) can also be used to generate either transgenic animals or “knock out” animals which, in turn, are useful in the development and screening of therapeutically useful reagents. A transgenic animal (e.g., a mouse, rat or fish) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, CDNA encoding foggy can be used to clone genomic DNA encoding foggy in accordance with established techniques and the genomic sequences used to generate transgenic animals that contain cells which express DNA encoding foggy. Methods for generating transgenic animals, particularly animals such as mice or rats, have become conventional in the art and are described, for example, in U.S. Pat. Nos. 4,736,866 and 4,870,009. Typically, particular cells would be targeted foggy transgene incorporation with tissue-specific enhancers. Transgenic animals that include a copy of a transgene encoding foggy introduced into the germ line of the animal at an embryonic stage can be used to examine the effect of increased expression of DNA encoding foggy. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of foggy can be used to construct a foggy “knock out” animal which has a defective or altered gene encoding foggy as a result of homologous recombination between the endogenous gene encoding foggy and altered genomic DNA encoding foggy introduced into an embryonic stem cell of the animal. For example, cDNA encoding foggy can be used to clone genomic DNA encoding foggy in accordance with established techniques. A portion of the genomic DNA encoding foggy can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the PRO polypeptide.

Nucleic acid encoding the PRO polypeptides may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., 1986, Proc. Natl. Acad. Sci. USA 83:4143-4146). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., 1993, Trends in Biotechnology 11, 205-210). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., 1987, J. Biol. Chem. 262, 4429-4432; and Wagner et al., Proc. Natl. Acad. Sci. USA, 1990, 87,3410-3414. For review of gene marking and gene therapy protocols see Anderson et al., 1992, Science 256, 808-813.

The foggy polypeptides described herein may also be employed as molecular weight markers for protein electrophoresis purposes and the isolated nucleic acid sequences may be used for recombinantly expressing those markers.

The nucleic acid molecules encoding the foggy polypeptides or fragments thereof described herein are useful for chromosome identification. In this regard, there exists an ongoing need to identify new chromosome markers, since relatively few chromosome marking reagents, based upon actual sequence data are presently available. Each foggy nucleic acid molecule of the present invention can be used as a chromosome marker.

The foggy polypeptides and nucleic acid molecules of the present invention may also be used diagnostically for tissue typing, wherein the foggy polypeptides of the present invention may be differentially expressed in one tissue as compared to another, preferably in a diseased tissue as compared to a normal tissue of the same tissue type. Foggy nucleic acid molecules will find use for generating probes for PCR, Northern analysis, Southern analysis and Western analysis.

The foggy polypeptides described herein may also be employed as therapeutic agents. The foggy polypeptides of the present invention can be formulated according to known methods to prepare pharmaceutically useful compositions, whereby the foggy product hereof is combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™ or PEG.

The formulations to be used for in vivo administration must be sterile. This is readily accomplished by filtration through sterile filtration membranes, prior to or following lyophilization and reconstitution.

Therapeutic compositions herein generally are placed into a container having a sterile access port, for example, an intravenous solution bag or vial having a stopper pierceable by a hypodermic injection needle.

The route of administration is in accord with known methods, e.g. injection or infusion by intravenous, intraperitoneal, intracerebral, intramuscular, intraocular, intraarterial or intralesional routes, topical administration, or by sustained release systems.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

When in vivo administration of a foggy polypeptide or agonist or antagonist thereof is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

Where sustained-release administration of a PRO polypeptide is desired in a formulation with release characteristics suitable for the treatment of any disease or disorder requiring administration of the PRO polypeptide, microencapsulation of the PRO polypeptide is contemplated. Microencapsulation of recombinant proteins for sustained release has been successfully performed with human growth hormone (rhGH), interferon-(rhIFN-), interleukin-2, and MN rgp120. Johnson et al, 1996, Nat. Med., 2:795-799 (1996); Yasuda, 1993, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

The sustained-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

This invention encompasses methods of screening compounds to identify those that mimic the foggy polypeptide (agonists) or prevent the effect of the foggy polypeptide (antagonists). Screening assays for antagonist drug candidates are designed to identify compounds that bind or complex with the foggy polypeptides encoded by the genes identified herein, or otherwise interfere with the interaction of the encoded polypeptides with other cellular proteins. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for antagonists are common in that they call for contacting the drug candidate with a foggy polypeptide encoded by a nucleic acid identified herein under conditions and for a time sufficient to allow these two components to interact.

In binding assays, the interaction is binding and the complex formed can be isolated or detected in the reaction mixture. In a particular embodiment, the foggy polypeptide encoded by the gene identified herein or the drug candidate is immobilized on a solid phase, e.g., on a microtiter plate, by covalent or non-covalent attachments.

Non-covalent attachment generally is accomplished by coating the solid surface with a solution of the foggy polypeptide and drying. Alternatively, an immobilized antibody, e.g., a monoclonal antibody, specific for the foggy polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular foggy polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, 1989, Nature 340:245-246; Chien et al., 1991, Proc. Natl. Acad. Sci. USA, 88:9578-9582) as disclosed by Chevray and Nathans, Proc. Natl. Acad. Sci. USA, 1991, 89: 5789-5793). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding a foggy polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the foggy polypeptide may be added to a cell along with the compound to be screened for a particular activity and the ability of the compound to inhibit the activity of interest in the presence of the foggy polypeptide indicates that the compound is an antagonist to the foggy polypeptide. Alternatively, antagonists may be detected by combining the foggy polypeptide and a potential antagonist with membrane-bound foggy polypeptide receptors or recombinant receptors under appropriate conditions for a competitive inhibition assay. The foggy polypeptide can be labeled, such as by radioactivity, such that the number of foggy polypeptide molecules bound to the receptor can be used to determine the effectiveness of the potential antagonist. The gene encoding the receptor can be identified by numerous methods known to those of skill in the art, for example, ligand panning and FACS sorting. Coligan et al., Current Protocols in Immunol., 1(2): Chapter 5 (1991). Preferably, expression cloning is employed wherein polyadenylated RNA is prepared from a cell responsive to the foggy polypeptide and a cDNA library created from this RNA is divided into pools and used to transfect COS cells or other cells that are not responsive to the foggy polypeptide. Transfected cells that are grown on glass slides are exposed to labeled foggy polypeptide. The foggy polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, labeled foggy polypeptide can be photoaffinity-linked with cell membrane or extract preparations that express the receptor molecule. Cross-linked material is resolved by PAGE and exposed to X-ray film. The labeled complex containing the receptor can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative receptor.

In another assay for antagonists, mammalian cells or a membrane preparation expressing the receptor would be incubated with labeled foggy polypeptide in the presence of the candidate compound. The ability of the compound to enhance or block this interaction could then be measured.

More specific examples of potential antagonists include an oligonucleotide that binds to the fusions of immunoglobulin with foggy polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related protein, for example, a mutated form of the foggy polypeptide that recognizes the receptor but imparts no effect, thereby competitively inhibiting the action of the foggy polypeptide.

Another potential foggy polypeptide antagonist is an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature foggy polypeptides herein, is used to design an antisense RNA oligonucleotide of from about 10 to 40 base pairs in length. A DNA oligonucleotide is designed to be complementary to a region of the gene involved in transcription (triple helix—see Lee et al., 1979, Nucl. Acids Res., 6:3073); Cooney et al., 1988, Science, 241: 456; Dervan et al., 1991, Science, 251:1360), thereby preventing transcription and the production of the foggy polypeptide. The antisense RNA oligonucleotide hybridizes to the mRNA in vivo and blocks translation of the mRNA molecule into the PRO polypeptide (antisense—Okano, 1991, Neurochem., 56:560); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the foggy polypeptide. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, the receptor binding site, or growth factor or other relevant binding site of the foggy polypeptide, thereby blocking the normal biological activity of the foggy polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, 1994, Current Biology, 4:469-471, and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

These small molecules can be identified by any one or more of the screening assays discussed hereinabove and/or by any other screening techniques well known for those skilled in the art.

Diagnostic and therapeutic uses of the herein disclosed molecules may also be based upon the positive functional assay hits disclosed and described below.

F. Anti-Foggy Antibodies

The present invention further provides anti-foggy antibodies. Exemplary antibodies include polyclonal, monoclonal, humanized, bispecific, and heteroconjugate antibodies.

1. Polyclonal Antibodies

The anti-foggy antibodies may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the foggy polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

2. Monoclonal Antibodies

The anti-foggy antibodies may, alternatively, be monoclonal antibodies. Monoclonal antibodies may be prepared using hybridoma methods, such as those described by Kohler and Milstein, 1975, Nature, 256:495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the foggy polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, 1984, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against foggy. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridomacells may be grown in vivo as ascites in a mammal.

The monoclonal antibodies secreted by the subelones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

3. Human and Humanized Antibodies

The anti-foggy antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fe), typically that of a human immunoglobulin [Jones et al., 1986, Nature, 321:522-525 (1986); Riechmann et al., 1988, Nature, 332:323-329; and Presta, 1992, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., 1986, Nature, 321:522-525); Riechmann et al., 1988, Nature, 332:323-327 (1988); Verhhoeyn et al., 1988, Science, 239:1534-1536], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, 1991, J. Mol. Biol., 227:381; Marks et al, 1991, J. Mol Biol, 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., 1991, J. Immunol., 147(1):86-95). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., 1992, Bio/Technology 10, 779-783); Lonberg et al., 1994, Nature 368, 856-859; Morrison, 1994, Nature 368, 812-13); Fishwild et al. 1996, Nature Biotechnology 14, 845-51; Neuberger, 1996, Nature Biotechnology 14, 826; Lonberg and Huszar, 1995, Intern. Rev. Immunol. 13 65-93.

The antibodies may also be affinity matured using known selection and/or mutagenesis methods as described above. Preferred affinity matured antibodies have an affinity which is five times, more preferably 10 times, even more preferably 20 or 30 times greater than the starting antibody (generally murine, humanized or human) from which the matured antibody is prepared.

4. Bispecific Antibodies

Bispecific antibodies are monoclonal, preferably human or humanized, antibodies that have binding specificities for at least two different antigens. In the present case, one of the binding specificities is for the foggy, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, 1983, Nature, 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., 1991, EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., 1986, Methods in Enzymology, 121:210 (1986).

According to another approach described in WO 96/2701 1, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared can be prepared using chemical linkage. Brennan et al., 1985, Science 229:81 describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., 1992, J. Exp. Med. 175:217-225 describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., 1992, J. Immunol. 148(5):1547-1553. The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., 1993, Proc. Natl. Acad. Sci. USA 90:6444-6448 has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., 1994, J. Immunol. 152:5368. Antibodies with more than two valencies are contemplated. For example, trispecific antibodies can be prepared. Tutt et al., 1991, J. Immunol. 147:60.

Exemplary bispecific antibodies may bind to two different epitopes on a given foggy polypeptide herein. Alternatively, an anti-foggy polypeptide arm may be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcyRII (CD32) and FcγRIII (CD16) so as to focus cellular defense mechanisms to the cell expressing the particular foggy polypeptide. Bispecific antibodies may also be used to localize cytotoxic agents to cells which express a particular foggy polypeptide. These antibodies possess a foggy-binding arm and an arm which binds a cytotoxic agent or a radionuclide chelator, such as EOTUBE, DPTA, DOTA, or TETA. Another bispecific antibody of interest binds the foggy polypeptide and further binds tissue factor (TF).

5. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

6. Effector Function Engineering

It may be desirable to modify the antibody of the invention with respect to effector function, so as to enhance, e.g., the effectiveness of the antibody in treating cancer. For example, cysteine residue(s) may be introduced into the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., 1992, J. Exp Med., 176: 1191-1195 and Shopes, 1992, J. Immunol., 148: 2918-2922. Homodimeric antibodies with enhanced anti-tumor activity may also be prepared using heterobifunctional cross-linkers as described in Wolff et al., 1993, Cancer Research, 53: 2560-2565 (1993). Alternatively, an antibody can be engineered that has dual Fc regions and may thereby have enhanced complement lysis and ADCC capabilities. See Stevenson et al., 1989, Anti-Cancer Drug Design, 3: 219-230 (1989).

7. Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., 1987, Science, 238: 1098. Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody may be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

8. Immunoliposomes

The antibodies disclosed herein may also be formulated as immunoliposomes. Liposomes containing the antibody are prepared by methods known in the art, such as described in Epstein et al., 1985, Proc. Natl. Acad. Sci. USA, 82: 3688; Hwang et al, 1980, Proc. Natl. Acad. Sci. USA, 77: 4030; and U.S. Pat. Nos. 4,485,045 and 4,544,545. Liposomes with enhanced circulation time are disclosed in U.S. Pat, No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., 1982, J. Biol. Chem., 257: 286-288 via a disulfide-interchange reaction. A chemotherapeutic agent (such as Doxorubicin) is optionally contained within the liposome. See Gabizon et al., 1989, J. National Cancer Inst., 81(19): 1484.

9. Pharmaceutical Compositions of Antibodies

Antibodies specifically binding a foggy polypeptide identified herein, as well as other molecules identified by the screening assays disclosed hereinbefore, can be administered for the treatment of various disorders in the form of pharmaceutical compositions.

If the foggy polypeptide is intracellular and whole antibodies are used as inhibitors, internalizing antibodies are preferred. However, lipofections or liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al, 1993, Proc. Natl. Acad. Sci. USA, 90: 7889-7893. The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

G. Uses for Anti-Foggy Antibodies

The anti-foggy antibodies of the invention have various utilities. For example, anti-foggy antibodies may be used in diagnostic assays for foggy, e.g., detecting its expression (and in some cases, differential expression) in specific cells, tissues, or serum. Various diagnostic assay techniques known in the art may be used, such as competitive binding assays, direct or indirect sandwich assays and immunoprecipitation assays conducted in either heterogeneous or homogeneous phases [Zola, Monoclonal Antibodies: A Manual of Techniques, CRC Press, Inc. (1987) pp. 147-158]. The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., 1962, Nature, 144:945; David et al., 1974, Biochemistry, 13:1014; Pain et al., 1981, J. Immunol. Meth., 40:219 (1981); and Nygren, 1982, J. Histochem. and Cytochem., 30:407.

Anti-foggy antibodies also are useful for the affinity purification of foggy from recombinant cell culture or natural sources. In this process, the antibodies against foggy are immobilized on a suitable support, such a Sephadex resin or filter paper, using methods well known in the art. The immobilized antibody then is contacted with a sample containing the foggy to be purified, and thereafter the support is washed with a suitable solvent that will remove substantially all the material in the sample except the foggy, which is bound to the immobilized antibody. Finally, the support is washed with another suitable solvent that will release the foggy from the antibody.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### I. Experimental Procedures

Fish Stocks and Maintenance

Fish breeding and maintaining were performed as described in Guo et al., supra. Embryos were raised at 28.5° C.h and staged according to Kimmel et al., 1995, Dev. Dynam. 203: 253-310.

In situ Hybridization and Immunostaining

RNA probes were synthesized from linearized templates using RNA labeling reagents (Boehringer). TH and 5HT antibodies were purchased from Chemicon. FLAG antibody was purchased from Kodak. Zn-8 (Trevarrow et al., 1990, Neuron 4: 669-79) and anti-Hu antibodies (Marusich et al., 1994, J. Neurobiol. 25: 143-55) were obtained from the University of Oregon. 3A10 monoclonal antibody, developed by Thomas Jessell, was obtained from the Developmental Studies Hybridoma Bank. In situ hybridization and antibody staining were performed as previously described. Guo et at, supra.

Isolation of Closely Linked DNA Polymorphisms by AFLP

Heterozygous female fish (AB/EK) carrying the foggy mutation were crossed with WT male fish from s different genetic background (Tu), and F1 progeny were raised to adulthood. The heterozygous F1 fish were identified by sibling pair-mating. The F2 progeny were collected from heterozygous mating and separated into two pools (each pool containing 40 individuals) based on the phenotype: Wildtype+heterozygous (WT+Het) and mutant (mt) pools. Genomic DNA was extracted from the pooled fish, digested with restriction enzymes EcoRI and MseI, ligated to synthesized DNA adapters, and used for PCR analysis. PCR primers (adapter+EcoR1+NNN, adapter+MseI+NNN) were synthesized and dye-labeled by the Genentech oligo synthesis facility, and PCR conditions were used as previously described. Vos et al., supra. PCR samples were run on ABI automated sequencer 310, and data output was analyzed using ABI Genescan software. The pool size of 40 allowed us to identify polymorphic markers that are within about 2.5 cMs to the foggy locus.

Genetic Mapping and Positional Cloning

Identified AFLP markers were used to test on foggy mutant individuals to establish more precise genetic distance: first, the linked AFLP fragments were gel purified, cloned and sequenced. Second, based on sequencing information, specific PCR primers were designed, and most AFLP markers could be converted to PCR fragment length polymorphism or single-stranded conformation polymorphism. AFLP markers were positioned onto the microsatellite genetic map. PCR primers corresponding to the two most closely lined AFLP markers were then used to screen a microarrayed BAC library (Genome Systems) and YAC library (Research Genetics) using standard PCR conditions. YAC and BAC DNA were isolated according to the manufacturer's instructions, and automated cycle sequencers (ABI) sequenced the ends. Specific PCR primers were designed according to the end sequences, tested using radiation hybrid panels (Geisler et al., 1999, Nature Genetics 23: 86-9; Hukriede et al., 1999, Proc. Natl. Acad. Scil USA 96: 9745-50) and used to continue screening the BAC library until the entire foggy region was covered with genomic DNA clones.

Transformation Rescue with BACs and cDNAs

DNA for individual BAC was injected at a concentration of 40-60 ng/μl into 1 to 4 cell stage zebrafish embryos derived from foggy heterozygous mating. Injected embryos were allowed to develop to 48 hours, examined under dissecting microscope, and subjected to immunohistochemistry with TH antibody. After examination and photographing of the staining patterns, genomic DNA was extracted from individual embryos, and used for genotyping with PCR primers tightly linked to the foggy locus. The BAC B108J11, which rescued the foggy mutant phenotype, was digested with EcoRI, random-primed with p32, and used to screen a 33-hour embryonic zebrafish cDNA library. Full-length cDNAs were cloned into the vector containing beta-actin promoter, injected into embryos at a concentration of 15-30 ng/μl, and assayed for rescue as described above.

Sequence Analysis and Mutation Detection

Sequence analysis was done using a Unix system and software developed by the Genentech Bioinformatics group. For mutation detection, gene specific primers were used to amplify genomic DNA from pools of about 5 foggy mutant and WT sibling embryos. PCR products from mutant and WT sibling embryos (three independent sets) were directly sequenced using automated cycle sequencers (ABI). cDNAs from mutant and WT sibling embryos were synthesized by RT-PCR and sequenced. Site-directed mutagenesis was done using reagents from Stratagene to introduce the single nucleotide change T→A. CDNA containing the introduced mutation was cloned after actin promoter and assayted form transformation rescue as described above.

Protein Expression in COS-7 Cells

COS-7 cells were plated at about 60% confluency in a 1-ml microtiter dish, and transfected with 1 μg plasmid containing FLAG-tagged WT foggy cDNA or mutant foggy CDNA using GIBCO-BRL reagents. After 24 hours, the cells were fixed and immunostained with anti-FLAG antibody.

Recombinant Proteins for Transcription Assay

An NdeI site was created at the 5′-end of the zfSpt5 ORF by PCR mutagenesis. The NdeI (partial)-NotI fragment containing the full-length, wild-type or mutant ORF was inserted into NdeI-BamHI sites of pET-14b (Novagen), yielding pET-afSpt5 WT and pET-zfSpt5 mt. Expression and purification of histidine-tagged zfSpt5 proteins were done as described. Yamaguchi et al., 1999, Cell 97: 41-51. Briefly, lysates were prepared from E. coli BL21 (DE3) strain transformed with pET-zfSpt5 mt, respectively. His-zfSpt5 proteins were purified form the supernatants by Ni-affinity chromatography, separated on SDS-polyacrylamide gel, and the full-length polypeptides were recovered from gel slices.

Transcription Assays

Depletion/add-back assays were performed as described. Wada et al., supra. Indicated amounts of hSpt4 and either hSpt5, or WT or mutant sfSpt5 were added back to HeLa nuclear extract (2 μl) immunodepleted using anti-DSIF p160/hSpt5 monoclonal antibody. PTF3-6C2AT (25 ng) or pSLG402 (125 ng, Lee and Green supra) were used for a template. After a 45 minute incubation, transcription was allowed to initiate for the indicated time by the addition of NTPs with or without 50 μM DRB. For pSLG402, low NTP concentrations (30 mM ATP, 30 mM GTP, 300 mM CTP, 2.5 mM UTP) were employed. G-free transcripts were purified and analyzed by 8% urea-PAGE.

### II. Results

The foggy Mutation Leads to Deficits or Surpluses of Distinct Neuronal Populations

Zebrafish mutants affecting neuronal development have been previously isolated in a genetic screen employing the neurotransmitter synthesis enzyme tyrosine hydroxylase (TH) as a molecular marker for multiple groups of DA and NA neurons. Guo et al., suipra. One of the mutants designated foggy appeared morphologically normal (FIG. 1A&B), but suffered irreversible deficits in DA neurons in the hypothalamus as early as 28 hpf (hours post fertilization) when such neurons first typically appear. The deficits became more apparent by 48 hpf, as shown by a reduction in the number of TH and DA neurons and a decrease in the level of TH in the few remaining DA cell bodies (FIG. 1C&D). In the zebrafish embryo, a group of 5HT neurons develop in close proximity to the hypothalamic DA neurons (compare FIS. 1E to FIG. 1C). This drew attention to the fate of 5HT neurons in the foggy mutant. In the WT fish, 5HT+ neurons first appear between 22-28 hpf. Subsequently, by 48 bpf, they constitute two discrete clusters of 5-7 neurons, which occupy the dorsal hypothalamus/posterior tuberculum, medial to the DA neurons (FIG. 1E, 1G). Surprisingly, whereas the number of TH+ DA neurons was reduced from about 17+/−3 to about 8+/−2 on each side of the foggy hypothalamus, the number of 5HT+ neurons was nearly from about 6+/−1 to about 15+/−2 at this location (FIGS. 1F, 1H). Interestingly, immunofluorescent double labeling for TH and 5HT in the foggy mutant revealed that some neurons are positive for both markers (FIG. 1H). Neurons that express both TH and 5HT were not found in WT embryos (FIG. 1G). Although we can not formally exclude the possibility that foggy independently affect DA and 5HT neurons, these findings suggest that hypothalamic DA and 5HT neurons are derived from common progenitors and that the foggy mutation may have disrupted cell fate decisions in this lineage. In contract to the hypothalamus, 5HT+ neurons in the foggy hindbrain developed normally (FIGS. 1I-J).

Another brain region where deficits were observed in TH+ DA neurons was the retina (FIGS. 2A-B). The retina contains six major classes of neurons that are sequentially specified from shared progenitors in response to extrinsic cues. Cepko, 1999, Curr. Opin. Neurobiol. 9: 37-46. The retinal ganglion neurons appear first, followed by cone photoreceptors, horizontal cells amacrine interneurons, rod photoreceptors and bipolar neurons. The deficits in TH+ DA amacrine interneurons warranted further examination or other neurons in the foggy mutant retina. The analysis revealed that foggy suffers deficits in redopsin+ cones (FIGS. 2C-D) and rhodopsin+ rod photoreceptors as well as in GABA+ amacrine interneurons (FIGS. 2E-F). Interestingly, the number ofganglion neurons, the first cell type that develops in the retina, appeared normal and may have even increased (FIGS. 2G-H). In contract to the retina, differentiation of the lens and optic nerve appeared normal in the foggy mutant embryo, as judged by the histology (not shown) and expression of γ-crystallin (FIGS. 2I-J). Histological analysis of the foggy retina revealed that at 48 hpf (not shown) the layered organization was overall normal and that at 72 hpf (FIGS. 2K-L) both photoreceptor and amacrine cells were normal.

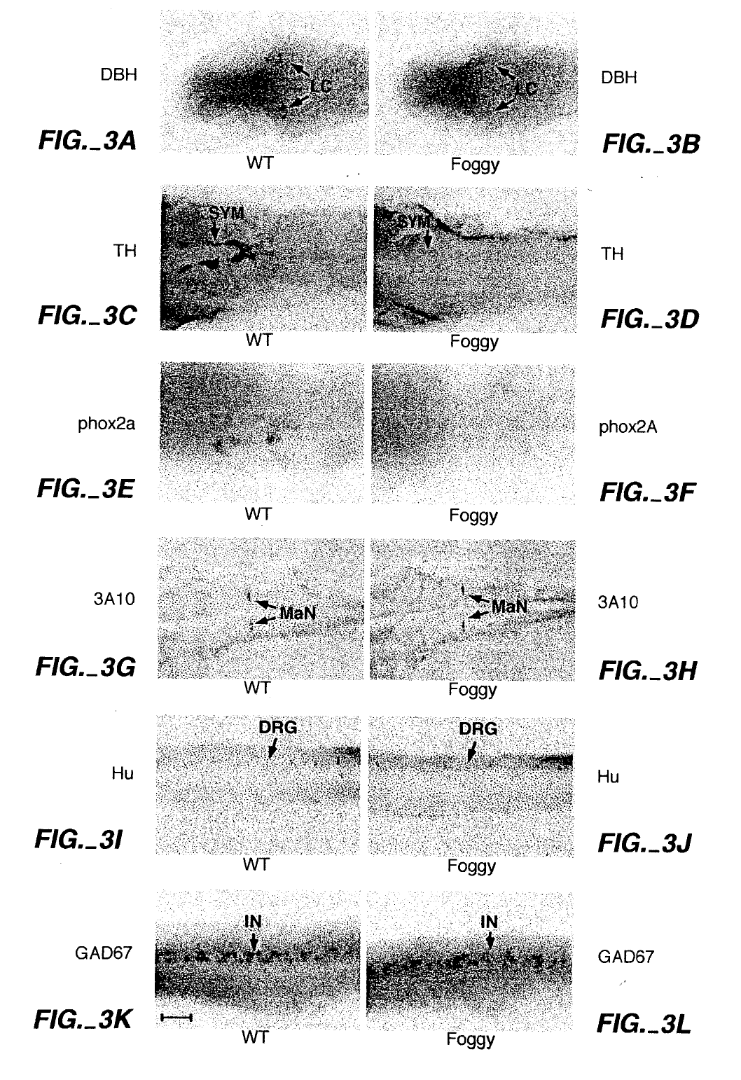

In addition, foggy failed to develop TH+ DBH+ NA neurons in the locus coeruleus (LC) of the hindbrain (FIGS. 3A-B), but possessed a normal complement of the hindbrain Mauthner neurons (FIGS. 3I-J), spinal GABA+ interneurons (FIGS. 3M-N) and spinal motoneurons (data not shown). The neural crest-derived sympathetic NA neurons (FIGS. 3C-D) are also absent, while the neural crest-derived dorsal root ganglia sensory neurons appear normal (FIGS. 3K-L). One group of NA cells, the TH+, DBH+ arch associated cells (AAC), which reside adjacent to the heart remained normal in the foggy mutant. Guo et al., supra. The transcription factor Phox2a that is essential for the acquisition of NA neuron identity (Guo et al., 1999, Neuron 24: 555-66; Morin et al., 1997, Neuron 18: 411-423), was normally expressed in LC early progenitors (FIGS. 3E-F). However, it was largely absent from the regions where the mature LC and sympathetic neurons should reside (FIGS. 3G-H and data not shown). However, no abnormal cell death was detected by tunnel staining of whole embryos at 24, 48, 60 and 72 hpf (data not shown). Outside of the nervous system, foggy embryos were indistinguishable from wildtype (WT) siblings by visual inspection at 28 hpf. At this developmental stage, they also displayed normal expression pattern of the regional markers Sonic hedgehog (Shh), fibroblast growth factor-8 (Fgf8), Otx1, Otx2 and Krox-20 (data show). The 48 hpf foggy embryos still possessed histologically normal notochord, spinal cord, somits, otic vesicles, eye, oral invagination, endoderm primordium of the liver, pronephros, yolk sac, heart, blood cells, pharyngeal arches, brain and brain ventricles (data not shown). By 72 hpf, while most tissues and organs remain normal, the embryos displayed reduction in neural crest-derived melanocytes, distended pericardial sac due to block of circulation, thin myocardial walls, smaller eyes and smaller body size (FIGS. 1A&B, 2M&N and data not shown). Taken together, the mutant phenotype is consistent with the idea that foggy is essential for normal development of multiple classes of neurons in the central and peripheral nervous system at a stage before they express neurotransmitter synthesis enzymes.

Isolation of the foggy Gene

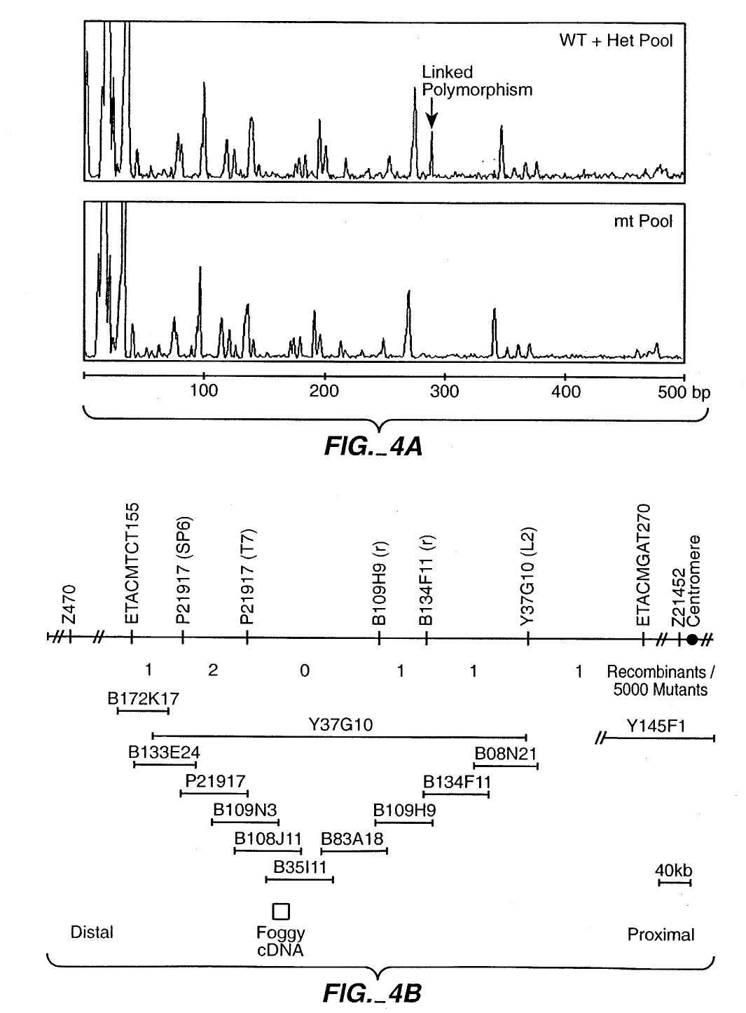

The foggy gene was isolated by the DNA fingerprinting technology called AFLP (Amplified Fragment Length Polymorphism, Vos et al., Nucl. Acids Res. 23: 4407-14), which allowed a rapid genome-wide search for polymorphic DNA markers closely linked to the foggy locus (FIG. 4A). Two AFLP markers, ETACMTCT155 and ETACMGAT270, were isolated and found to be tightly linked to the foggy locus (0 recombinant out of 200 individual mutants tested). Further lineage analysis of about 5000 individual mutants established that each AFLP marker was 0.06+/−0.02 cM away on either side of the foggy locus. During the course of our AFLP analysis, a genetic map consisting of 2000 microsatellite markers were published. Shimoda et al., 1999, Genomics 58:219-32. AFLP markers were positioned on this map and assigned the foggy locus to Chromosome 15. Unfortunately, all the available microsatellite markers in this region of chromosome 15 were mapped at least 1 cM away form the foggy locus. (FIG. 4B). Thus, by AFLP, genetic mapping and linkage analysis we have identified the interval of foggy locus as existing between the polymorphic DNA markers ETACMTCTT155 and ETACMGAT270, and determined that it bears a genetic distance of 0.12+/−0.04 cM.

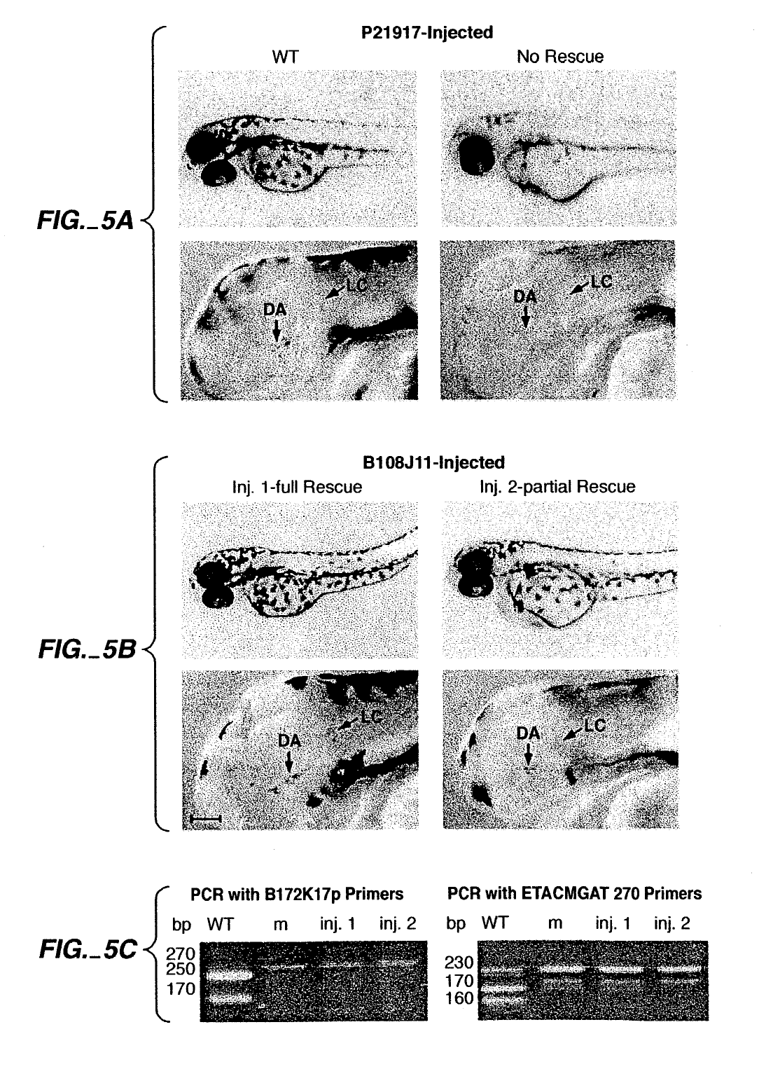

Genomic clones composed of Yeast Artificial Chromosomes (YACs), BACs and PACs were isolated using the two AFLP markers. The ends of these large DNA clones were sequenced, and used as markers to isolate additional clones that span the entire foggy locus (FIG. 4B). Fine lineage analysis was performed using PCR Fragment Length Polymorphisms (PFLP) or Single-Stranded Conformation Polymorphisms (SSCP) that were identified by BAC/PAC end sequencing. This analysis allowed us to locate the foggy locus to an interval spanned by four BACs. To identify which Bac contains the foggy gene, rescue experiments were performed as described in Yan et al., 1998, Genomics 50: 287-9. BAC clones (Table 1) were injected into one or two-cell stage zebrafish embryos that were derived from foggy heterozygous matings, and the embryos were analyzed by immunohistochemistry with TH antibody for the appearance of TH+ DA and NA neurons at about 48 hpf. BAC B108J11 and BAC B 35I11 were able to restore the normal development of melanocytes and TH+ DA and NA neurons to homozygous foggy mutants (FIG. 5B, Table 1 and data not shown), suggesting that they contain the WT foggy gene. Shotgun sequencing of the entire BAC B108J11 is likely to be the foggy gene (Table 1). To further test this possibility, we isolated a full-length cDNA corresponding to this transcription unit, cloned the cDNA into a vector containing the zebrafish betaactin promoter (Higashijima et al., 1997, Devel. Biol. 192: 289-99) and injected the construct into zebrafish embryos. The cDNA was able to restore the normal development of the foggy mutant embryos (Table 1), providing strong evidence that this transcription unit corresponds to the foggy gene. Ectopic expression of this cDNA did not interfere with the development of WT embryos (data not shown).

Foggy Encodes a Widely Expressed Nuclear Protein Homologous to Spt5, a Regulator of Transcription Elongation

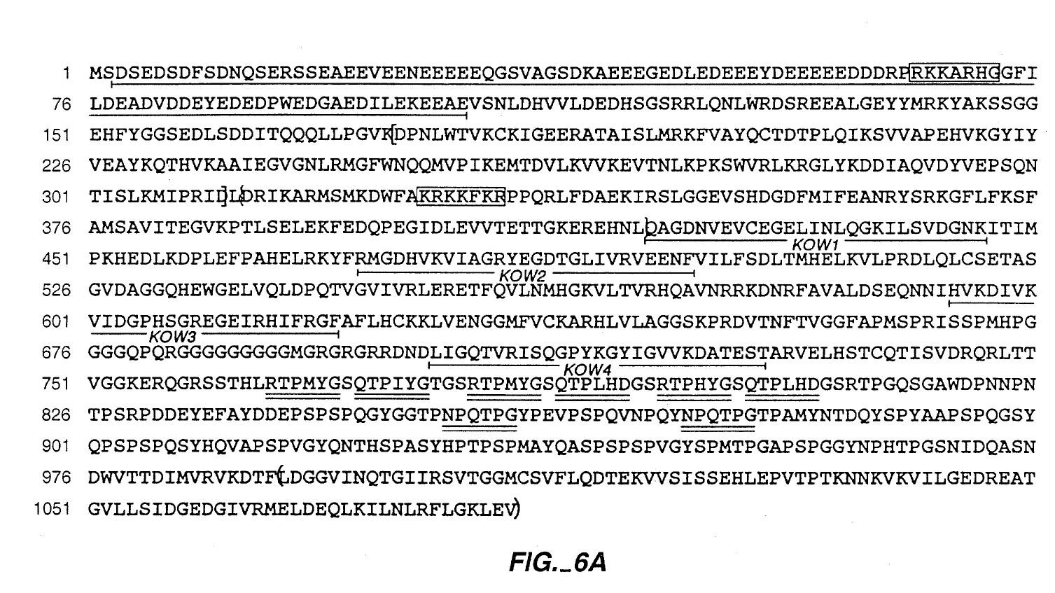

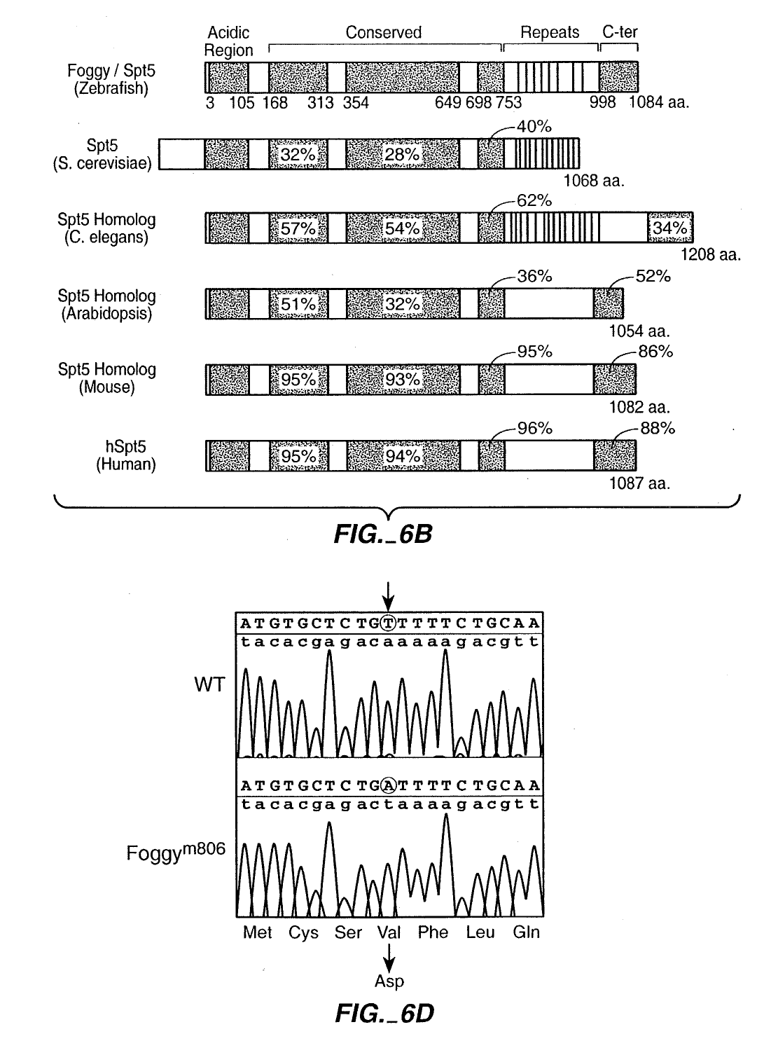

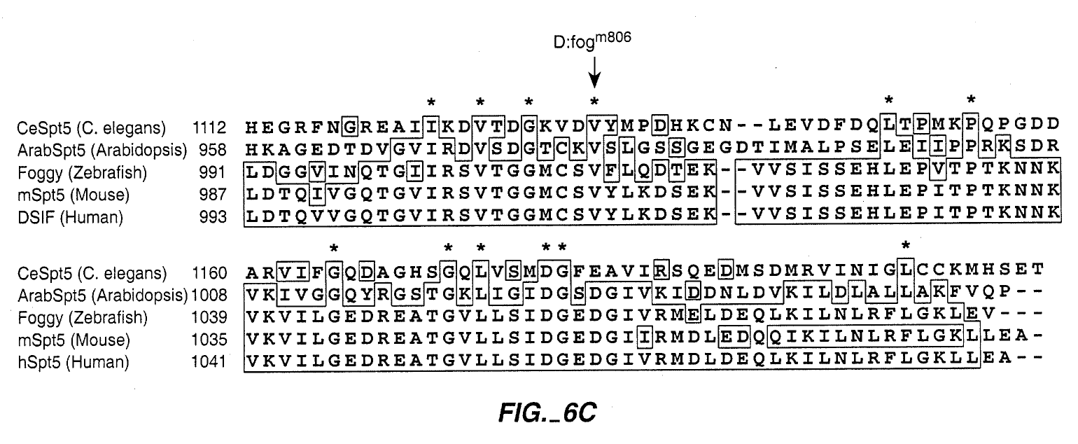

DNA sequence analysis of the rescuing cDNA and database searches revealed that the putative foggy protein contains 1084 amino acid residues (FIG. 6A), and belongs to an evolutionarily conserved family of proteins (FIG. 6B) having homology to the yeast Spt5. Swanson et al., supra. Although in vitro studies suggested this protein family functions as both positive and negative regulators of transcription elongation, no evidence for their role in vivo was available in vertebrates. Foggy contains an N-terminal acidic region of 105 amino acids, 53 of which are either Asp or Glu (FIG. 6A) and its central domain is composed of four NusB/KOW domains having an unknown function. Similar NusG/KOW domains are found in NusG, a prokaryotic factor that regulates transcription elongation (Sullivan and Gottesman, 1992, Cell 68: 989-94; Sullivan et al., 1992, J. Bact. 174: 1339-44) and in a class of proteins involved in translation. Kyrpides et al., 1996, Trends Biochem. Sci. 21: 425-6. The carboxyl-terminus of foggy is rich in the amino acids serine, threonine, and tyrosine, which form potential phosphorylation sites and displays three types of hexapeptide repeats: 4× XTPXYG, 2× QTPLHD and 2× NPQTPG. The C-terminal end (˜100 amino acids) are conserved among all multicellular organisms, but are absent in the yeast Spt5 (FIG. 6C). Sequence analysis of genomic DNA and cDNA prepared from foggy mutants and their WT siblings identified a single nucleotide change from T to A, which changed the encoded amino acid from valine-1012 to aspartic acid (FIG. 6D). The valine-1012 is an absolutely conserved amino acid amongst various cross-species members of this protein family, and is located in the conserved C-terminal domain (FIG. 6C). To further discern whether the valine-1012 to aspartic acid mutation indeed has functional consequences and does not represent an inert polymorphism, a construct carrying the mutation was injected into zebrafish embryos and its ability to rescue the mutant phenotype was assessed. Whereas the WT foggy CDNA readily rescued the mutant phenotype, the construct bearing the single nucleotide change had no effect under the same conditions (FIG. 1). Taken together, these findings demonstrate that the isolated CDNA indeed encodes for foggy and that the single nucleotide change is responsible for the foggy mutant phenotype.

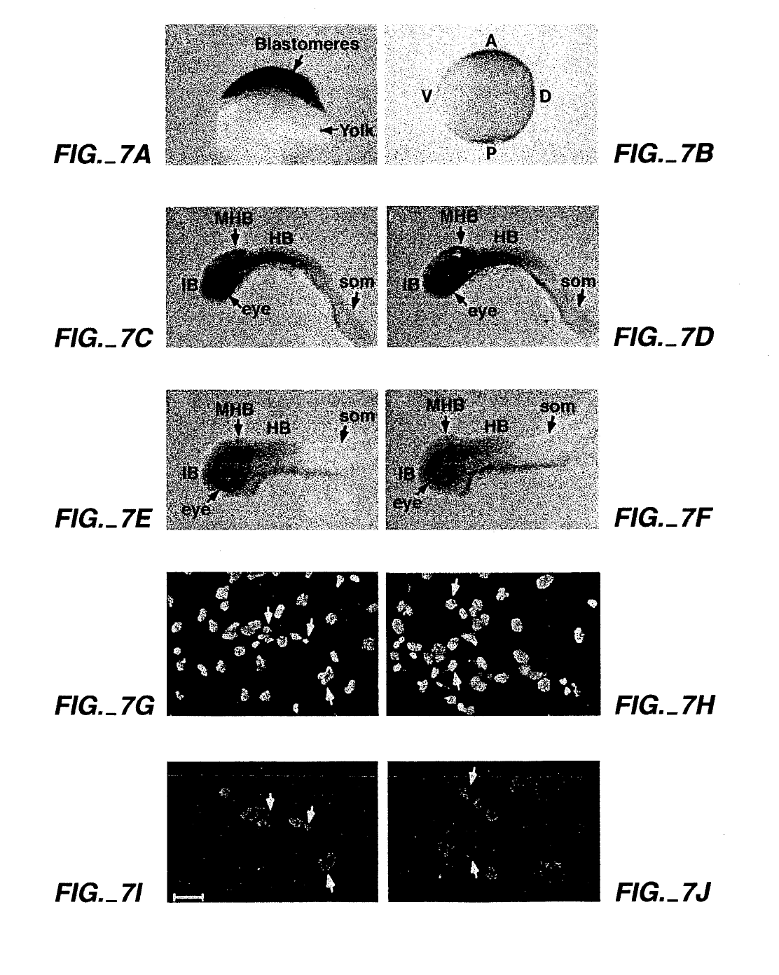

To begin characterizing foggy, the spatial and temporal expression pattern during development were examined. By whole mount in situ hybridization, maternal foggy mRNA was detected in all blastomeres of early embryos (FIG. 7A). In the tailbud-stage embryo (about 10 hpf), foggy expression is concentrated in the dorsal neural plate with low level expression in the ventral epidermis (FIG. 7B). At about 28 hpf, foggy mRNA is detected predominantly in the developing brain with low level expression elsewhere (FIG. 7C), and its expression pattern is not altered in the foggy mutant embryos (FIG. 7D). By 48 hpf, there is an apparent down-regulation of foggy transcripts throughout the embryo (compare FIG. 7C to 7E). To determine whether the foggy protein can enter the nucleus and whether the foggy mutation disrupts its nuclear access, we expressed epitope-tagged WT and mutant proteins in mammalian Cos-7 cells, and their sub-cellular distribution was examined by immunofluorescent labeling. As shown in FIGS. 7G-J, both WT and the Val-1012 to Asp mutant forms of foggy were detected and the cell nucleus, suggesting that foggy is a nuclear protein, and that the mutation did not disrupt the protein access to the nucleus.

Foggy Regulates Transcription Elongation in vitro and the Mutant Form is Selectively Inactive as a Repressor

Also analyzed was whether the WT foggy was indeed a functional homolog of Spt5 and if so, whether the val-1012 to Asp mutation disrupts its transcriptional activity. The assay used was the Spt4/Spt5-dependent inhibition of transcription elongation by transcription elongation inhibitory compound DRB in a crude Hela nuclear extract. Wada et al., 1998, supra. WT and mutant foggy that were expressed in and purified from E. coli appeared identical to their human counterpart and to each other in size and mobility (FIG. 8A). Moreover, WT foggy was as effective as the hSpt5 in restoring the response to drug DRB (FIG. 8B, lanes 5 and 6) supporting the notion that these two proteins are indeed functional homologs. In contrast, mutant foggy failed to confer elongation inhibitory drug DRB -sensitivity, even when added at 9-fold higher concentrations (FIG. 8B, lanes 7-12). These data suggest that foggy can biochemically act as a repressor of transcription elongation, and the 1012-Val to Asp mutations abolishes this activity.

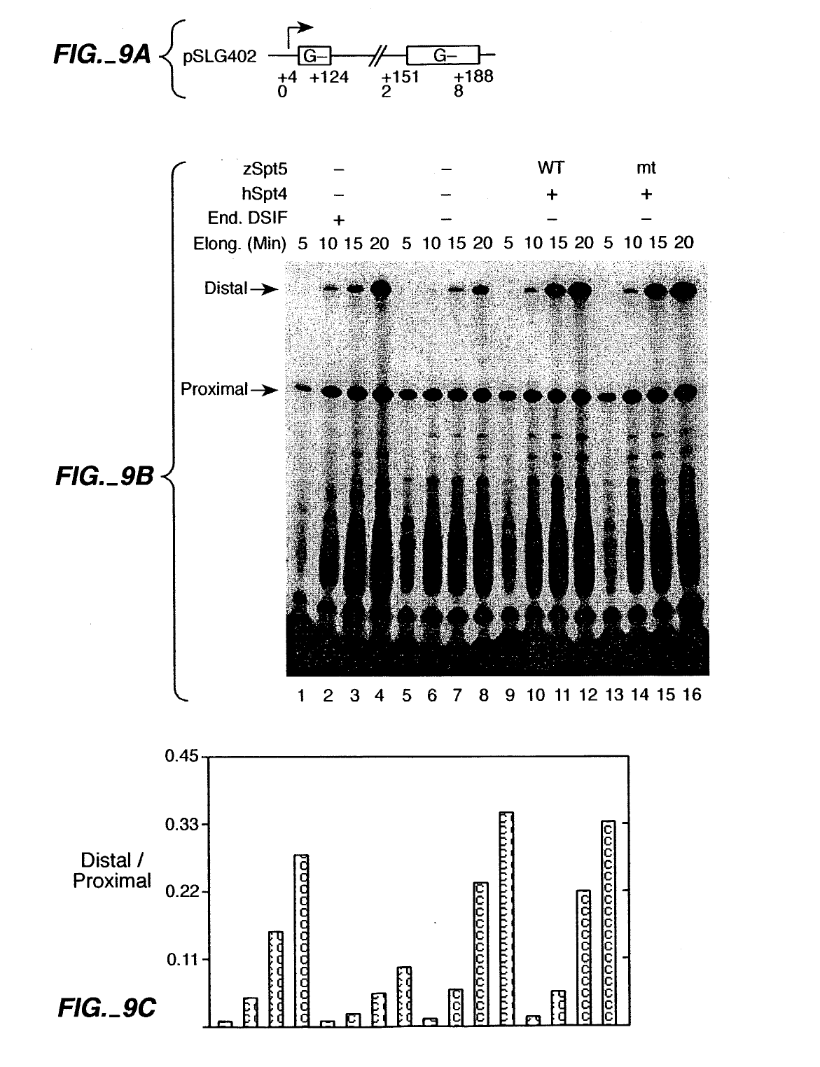

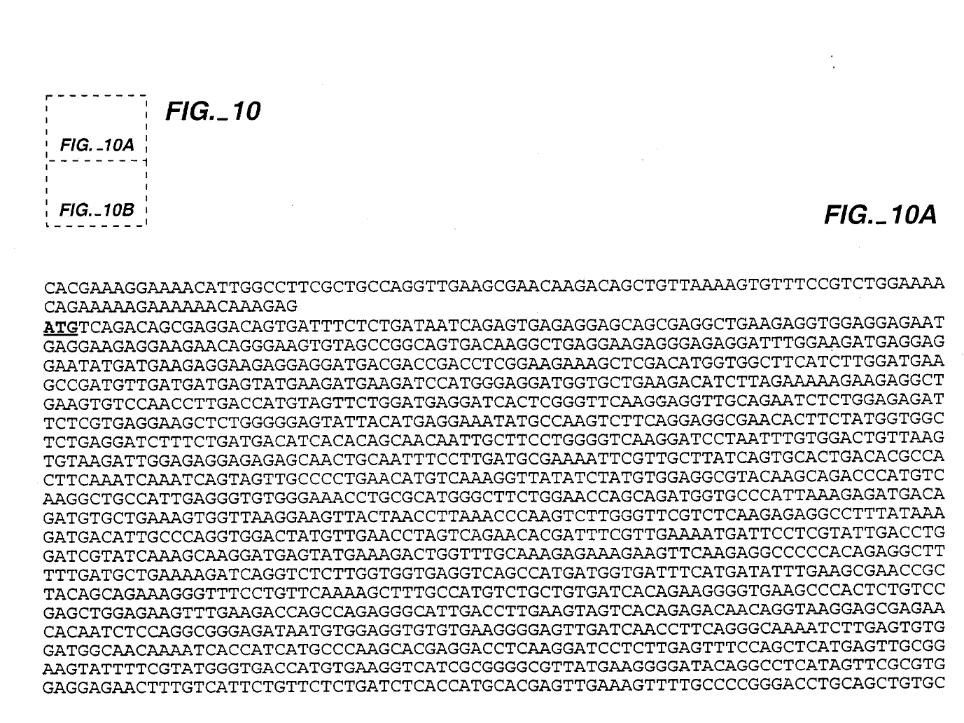

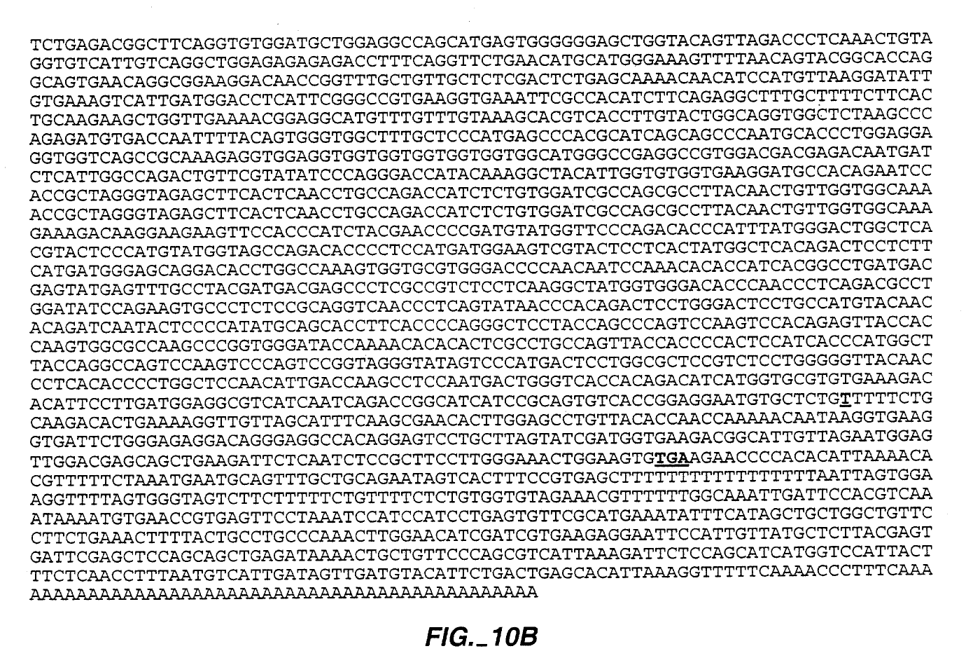

As mentioned previously, low concentrations of Spt4/Spt5 (DSIF) complex stimulates rather than represses transcription elongation. Wada et al.,supra. A DNA template was used to quantitatively measure possible stimulatory activities of the WT and mutant zSpt5, (Lee and Greenleaf, 1997, J. Biol. Chem. 272: 10990-10993), which contains two G-free cassettes, a promoter-proximal 85-nt cassette at positions +40 to +124, and a promoter distal 377-nt cassette at positions +1512 to +1888. These two cassettes are resistant to RNase T1 digestion and are separated by G-rich, RNase T1 sensitive region. Following transcription by RNA Pol II and RNase T1 digestion, the two cassettes are released from the transcripts and can be separated by gel electrophoresis. Their relative quantity reflects the elongation efficiency of transcription. When normal nuclear extract was used, the molar ratio between the distal to proximal regions of the transcript increased with time, approaching a value 0.5 (FIG. 9, lanes 1-4; Lee and Greenleaf, supra). Depletion of DSIF from the extract resulted in a significant reduction in elongation efficiency. FIGS. 9C-D, lanes 5-8. Reconstitution of the depleted extract with rDSIF using either human or zebrafish Spt5 increased the efficiency of transcription elongation by 4.6 fold at 15 minutes an by 3.8 fold at 20 min. and restored it to the level of non-depleted extract (FIGS. 9C-D, and data not shown). Surprisingly, the mutant zSpt5 was as efficient as WT zSpt5 in stimulating elongation in this assay, increasing the efficiency of elongation by 4.3-fold at 15 minutes and by 3.6 fold at 20 minutes (FIGS. 9C-D, lanes 9-16). together, these results indicate that under these conditions, the mutant foggy/zSpt5 lost its ability to act as a negative but not as a positive regulator of transcription elongation.

### Example 2

### Expression of Foggy in E. coli

This example illustrates preparation of an unglycosylated form of a foggy polypeptide or foggy polypeptide antagonist polypeptide (“foggy”) by recombinant expression in E. coli.

The DNA sequence encoding foggy is initially amplified using selected PCR primers. The primers should contain restriction enzyme sites that correspond to the restriction enzyme sites on the selected expression vector. A variety of expression vectors may be employed. An example of a suitable vector is pBR322 (derived from E. coli; see Bolivar et al., 1977, Gene, 2:95) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the PRO coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized foggy protein can then be purified using a metal chelating column under conditions that allow tight binding of the protein.

Foggy may be expressed in E. coli in a poly-His tagged form, using the following procedure. The DNA encoding foggy is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) Ion galE rpoHts(htpRts) clpP(laclq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₄)₂SO₄, 0.71 g sodium citrate•2H2O, 1.07 g KCl, 5.36 g Difco yeast extract, 5.36 g Sheffield hycase SF in 500 mL water, as well as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1 M and 0.02 M, respectively, and the solution is stirred overnight at 4° C. This step results in denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded foggy are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

### Example 3

### Expression of PRO in Mammalian Cells

This example illustrates preparation of a potentially glycosylated form of foggy polypeptide and foggy polypeptide antagonist (“foggy”) by recombinant expression in mammalian cells.

The vector, pRK5 (see EP 307,247, published Mar. 15, 1989), is employed as the expression vector. Optionally, the foggy DNA is ligated into pRK5 with selected restriction enzymes to allow insertion of the foggy DNA using ligation methods such as described in Sambrook et al., supra. The resulting vector is called pRK5-foggy.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-foggy DNA is mixed with about 1 μg DNA encoding the VA RNA gene (Thimmappaya et al., 1982, Cell, 31 :543) and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of PRO polypeptide. The cultures containing transfected cells may undergo further incubation (in serum free medium) and the medium is tested in selected bioassays.

In an alternative technique, foggy may be introduced into 293 cells transiently using the dextran sulfate method described by Somparyrac et al., 1981, Proc. Natl. Acad. Sci., 12:7575 (1981). 293 cells are grown to maximal density in a spinner flask and 700 μg pRK5-foggy DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed foggy can then be concentrated and purified by any selected method, such as dialysis and/or column chromatography.

In another embodiment, foggy can be expressed in CHO cells. The pRK5-foggy can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of foggy polypeptide, the culture medium may be replaced with serum free medium. Preferably, the cultures are incubated for about 6 days, and then the conditioned medium is harvested. The medium containing the expressed foggy can then be concentrated and purified by any selected method.

Epitope-tagged foggy may also be expressed in host CHO cells. The foggy may be subcloned out of the pRK5 vector. The subclone insert can undergo PCR to fuse in frame with a selected epitope tag such as a poly-his tag into a Baculovirus expression vector. The poly-his tagged foggy insert can then be subcloned into a SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-His tagged foggy can then be concentrated and purified by any selected method, such as by Ni²⁺-chelate affinity chromatography.

Foggy may also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., 1996, Nucl. Acids Res. 24:9 (1774-1779), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Quiagen), Dosper® or Fugene® (Boehringer Mannheim). The cells are grown as described in Lucas et al., supra. Approximately 3×10⁻⁷ cells are frozen in an ampule for further growth and production as described below.

The ampules containing the plasmid DNA are thawed by placement into water bath and mixed by vortexing. The contents are pipetted into a centrifuge tube containing 10 mLs of media and centrifuged at 1000 rpm for 5 minutes. The supernatant is aspirated and the cells are resuspended in 10 mL of selective media (0.2 μm filtered PS20 with 5% 0.2 μm diafiltered fetal bovine serum). The cells are then aliquoted into a 100 mL spinner containing 90 mL of selective media. After 1-2 days, the cells are transferred into a 250 mL spinner filled with 150 mL selective growth medium and incubated at 37° C. After another 2-3 days, 250 mL, 500 mL and 200 ml spinners are seeded with 3×10⁵ cells/mL. The cell media is exchanged with fresh media by centrifugation and resuspension in production medium. Although any suitable CHO media may be employed, a production medium described in U.S. Pat. No. 5,122,469, issued Jun. 16, 1992 may actually be used. A 3L production spinner is seeded at 1.2×10⁶ cells/mL. On day 0, the cell number pH ie determined. On day 1, the spinner is sampled and sparging with filtered air is commenced. On day 2, the spinner is sampled, the temperature shifted to 33° C., and 30 mL of 500 g/L glucose and 0.6 mL of 10% antifoam (e.g., 35% polydimethylsiloxane emulsion, Dow Corning 365 Medical Grade Emulsion) taken. Throughout the production, the pH is adjusted as necessary to keep it at around 7.2. After 10 days, or until the viability dropped below 70%, the cell culture is harvested by centrifugation and filtering through a 0.22 μm filter. The filtrate was either stored at 4° C. or immediately loaded onto columns for purification.

For the poly-His tagged constructs, the proteins are purified using a Ni-NTA column (Qiagen). Before purification, imidazole is added to the conditioned media to a concentration of 5 mM. The conditioned media is pumped onto a 6 ml Ni-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc-containing) constructs are purified from the conditioned media as follows. The conditioned medium is pumped onto a 5 ml Protein A column (Pharmacia) which had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μL of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

Many of the PRO polypeptides disclosed herein were successfully expressed as described above.

### Example 4

### Expression of Foggy in Yeast

The following method describes recombinant expression of foggy and foggy polypeptide antagonist (“foggy”) in yeast.

First, yeast expression vectors are constructed for intracellular production or secretion of foggy from the ADH2/GAPDH promoter. DNA encoding foggy and the promoter is inserted into suitable restriction enzyme sites in the selected plasmid to direct intracellular expression of foggy. For secretion, DNA encoding foggy can be cloned into the selected plasmid, together with DNA encoding the ADH2/GAPDH promoter, a native foggy signal peptide or other mammalian signal peptide, or, for example, a yeast alpha-factor or invertase secretory signal/leader sequence, and linker sequences (if needed) for expression of foggy.

Yeast cells, such as yeast strain AB10, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant foggy can subsequently be isolated and purified by removing the yeast cells from the fermentation medium by centrifugation and then concentrating the medium using selected cartridge filters. The concentrate containing foggy may further be purified using selected column chromatography resins.

Many of the foggy polypeptides disclosed herein were successfully expressed as described above.

### Example 5

### Expression of Foggy in Baculovirus-Infected Insect Cells

The following method describes recombinant expression of foggy polypeptide and foggy polypeptide antagonist (“foggy”) in Baculovirus-infected insect cells.

The sequence coding for foggy is fused upstream of an epitope tag contained within a baculovirus expression vector. Such epitope tags include poly-his tags and immunoglobulin tags (like Fe regions of IgG). A variety of plasmids may be employed, including plasmids derived from commercially available plasmids such as pVL1393 (Novagen). Briefly, the sequence encoding foggy or the desired portion of the coding sequence of foggy such as the sequence encoding the extracellular domain of a transmembrane protein or the sequence encoding the mature protein if the protein is extracellular is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Laboratory Manual, Oxford: Oxford University Press (1994).

Expressed poly-his tagged PRO can then be purified, for example, by Ni²⁺-chelate affinity chromatography as follows. Extracts are prepared from recombinant virus-infected Sf9 cells as described by Rupert et al., 1993, Nature, 362:175-179. Briefly, Sf9 cells are washed, resuspended in sonication buffer (25 mLHepes, pH 7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted His₁₀-tagged PRO are pooled and dialyzed against loading buffer.

Alternatively, purification of the IgG tagged (or Fe tagged) PRO can be performed using known chromatography techniques, including for instance, Protein A or protein G column chromatography.

### Example 6

### Preparation of Antibodies that Bind Foggy

This example illustrates preparation of monoclonal antibodies which can specifically bind foggy polypeptide and foggy polypeptide antagonist (“foggy”).

Techniques for producing the monoclonal antibodies are known in the art and are described, for instance, in Goding, supra. Immunogens that may be employed include purified foggy, fusion proteins containing foggy, and cells expressing recombinant foggy on the cell surface. Selection of the immunogen can be made by the skilled artisan without undue experimentation.

Mice, such as Balb/c, are immunized with the foggy immunogen emulsified in complete Freund's adjuvant and injected subcutaneously or intraperitoneally in an amount from 1-100 micrograms. Alternatively, the iminunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-foggy antibodies.

After a suitable antibody titer has been detected, the animals “positive” for antibodies can be injected with a final intravenous injection of foggy. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against foggy. Determination of “positive” hybridoma cells secreting the desired monoclonal antibodies against foggy is within the skill in the art.

The positive hybridoma cells can be injected intraperitoneally into syngeneic Balb/c mice to produce ascites containing the anti-foggy monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 7

### Purification of Foggy Polypeptides Using Specific Antibodies

Native or recombinant foggy polypeptides and foggy polypeptide antagonists (“foggy”) may be purified by a variety of standard techniques in the art of protein purification. For example foggy is purified by immunoaffinity chromatography using antibodies specific for the foggy polypeptide of interest. In general, an immunoaffinity column is constructed by covalently coupling the anti-foggy polypeptide antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE (Pharmacia LKB Biotechnology). The antibody is coupled to the resin, the resin is blocked, and the derivative resin is washed according to the manufacturer's instructions.

Such an immunoaffinity column is utilized in the purification of foggy polypeptide by preparing a fraction from cells containingfoggy polypeptide in a soluble form. This preparation is derived by solubilization of the whole cell or of a subcellular fraction obtained via differential centrifugation by the addition of detergent or by other methods well known in the art. Alternatively, soluble foggy polypeptide containing a signal sequence may be secreted in useful quantity into the medium in which the cells are grown.

A soluble foggy polypeptide-containing preparation is passed over the immunoaffinity column, and the column is washed under conditions that allow the preferential absorbance of foggy polypeptide (e.g., high ionic strength buffers in the presence of detergent). Then, the column is eluted under conditions that disrupt antibody/foggy polypeptide binding (e.g., a low pH buffer such as approximately pH 2-3, or a high concentration of a chaotrope such as urea or thiocyanate ion), and foggy polypeptide is collected.

### Example 8

### Drug Screening

This invention is particularly useful for screening compounds by using foggy poypeptide or foggy polypeptide antagonist or binding fragment thereof (“foggy”) in any of a variety of drug screening techniques. The foggy employed in such a test may either be free in solution, affixed to a solid support, borne on a cell surface, or located intracellularly. One method of drug screening utilizes eukaryotic or prokaryotic host cells which are stably transformed with recombinant nucleic acids expressing foggy. Drugs are screened against such transformed cells in competitive binding assays. Such cells, either in viable or fixed form, can be used for standard binding assays. One may measure, for example, the formation of complexes between foggy and the agent being tested. Alternatively, one can examine the diminution in complex formation between foggy and its target cell or target receptors caused by the agent being tested.

Thus, the present invention provides methods of screening for drugs or any other agents which can affect a foggy—associated disease or disorder. These methods comprise contacting such an agent with foggy and assaying (i) for the presence of a complex between the agent and the foggy, or (ii) for the presence of a complex between foggy and the cell, by methods well known in the art. In such competitive binding assays, foggy is typically labeled. After suitable incubation, free foggy is separated from that present in bound form, and the amount of free or uncomplexed label is a measure of the ability of the particular agent to bind to foggy or to interfere with the foggy/cell complex.

Another technique for drug screening provides high throughput screening for compounds having suitable binding affinity to a polypeptide and is described in detail in WO 84/03564, published on Sep. 13, 1984. Briefly stated, large numbers of different small peptide test compounds are synthesized on a solid substrate, such as plastic pins or some other surface. As applied to a foggy polypeptide, the peptide test compounds are reacted with foggy and washed. Bound foggy polypeptide is detected by methods well known in the art. Purified foggy can also be coated directly onto plates for use in the aforementioned drug screening techniques. In addition, non-neutralizing antibodies can be used to capture the peptide and immobilize it on the solid support.

This invention also contemplates the use of competitive drug screening assays in which neutralizing antibodies capable of binding foggy specifically compete with a test compound for binding to foggy polypeptide or fragments thereof. In this manner, the antibodies can be used to detect the presence of any peptide which shares one or more antigenic determinants with foggy polypeptide.

What is claimed is:

1. A method of forming dopaminergic neurons by contacting neuroprogenitor cells in vitro with an effective amount of a foggy polypeptide.

2. The method of claim wherein the contacting occurs in vitro.

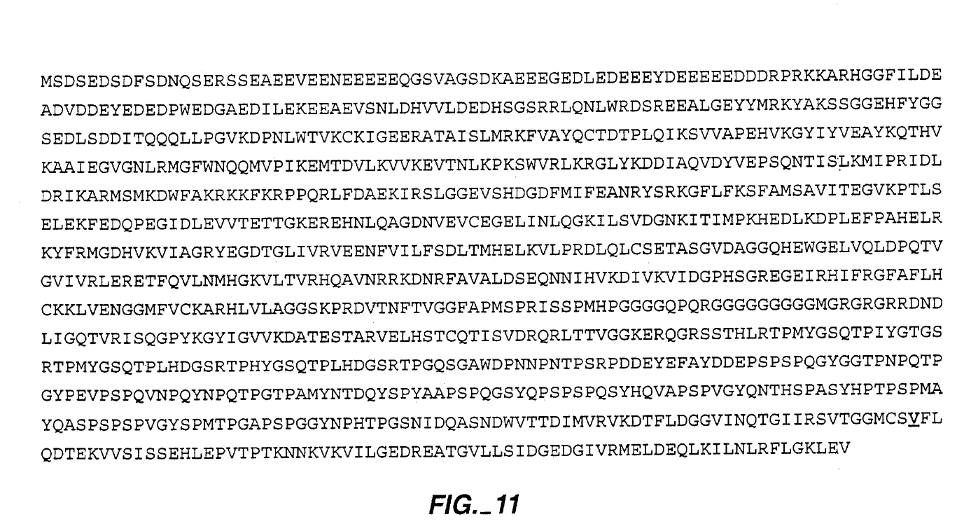

3. The method of claim 1 wherein the foggy polypeptide is active and is encoded by a nucleic acid having at least 80% nucleic acid sequence identity to a nucleic acid sequence encoding the amino acid sequence of FIG. 11 (SEQ ID NO:1).

4. The method of claim 1 wherein the foggy polypeptide comprises an active foggy amino acid sequence that has at least 80% amino acid sequence identity of FIG. 11 (SEQ ID NO:1).

5. The method of claim 1 wherein the foggy polypeptide is SEQ ID NO:1.

6. A method of forming serotonergic neurons by contacting neuroprogenitor cells in vitro with an effective amount of a foggy polypeptide antagonist.

7. The method of claim 6 wherein the contacting occurs in vitro.

8. The method of claim 6 wherein the foggy polypeptide antagonist is active and is encoded by a nucleic acid having at least 80% nucleic acid sequence identity to a nucleic acid sequence encoding the amino acid sequence of SEQ ID NO:3.

9. The method of claim 6 wherein the foggy polypeptide antagonist comprises an amino acid sequence that is an active foggy antagonist that has at least 80% amino acid sequence identity to SEQ ID NO:3.

10. The method of claim 6 wherein the foggy polypeptide is SEQ ID NO:3.

11. A method of treating a disorder in a mammal wherein said disorder is characterised by degeneration of dopaminergic neurons, comprising transplanting into said mammal a therapeutically effective amount of neuroprogenitor cells pretreated with an effective amount of a foggy polypeptide.

12. A method of treating a disorder in a mammal wherein said disorder is characterized by degeneration of serotonergic neurons, comprising transplanting into said mammal a therapeutically effective amount of neuroprogenitor cells pretreated with an effective amount of a foggy polypeptide antagonist.

13. The method of claim 11 further comprising the administration of a therapeutically effective amount of at least one neuronal survival factor.

14. The method of claim 13 wherein the neural survival factor is selected from the group consisting of: nerve growth factor (NGF); ciliary neurotrophic factor (CNTF), brain derived neurotrophic factor (BDNF), neurotrophin-3 (NT-3); neurotrophin-4 (NT-4); aFGF; IL-1β, TNF-(α, insulin-like growth factor (IGF-1, IGF-2), transforming growth factor beta (TGF-β, TGF-β1) and skeletal muscle extract.

15. The method of claim 11, wherein the disorder is one characterized by abnormalities in the regulation of postural reflexes, movement and reward-associated behaviors.

16. The method of claim 11, wherein the disorder is selected from the group consisting of: Parkinson's disease, schizophrenia, drug addiction and a condition caused by trauma or illness resulting in resting tremor, rigidity, akinesia or postural abnormality.

17. The method of claim 16, wherein the disorder is selected from the group consisting of adipsia, aphagia or sensory neglect.

18. The method of claim 12 further comprising the administration of a therapeutically effective amount of a neuronal survival factor.

19. The method of claim 12, wherein the disorder is one characterized by an abnormal regulation of food intake, hormone secretion, stress response, pain and immune function, sexual activity, cardiovascular function and temperature regulation.

20. The method of claim 12, wherein the disorder is selected from the group consisting of: depression; proclivity to suicide; violent aggressive behavior; obsessive-compulsive behavior; anorexia/bulimia and schizophrenia.

21. A composition of matter comprising neuroprogenitor cells and an effective amount of a foggy polypeptide antagonist.

22. A medical device comprising neuroprogenitor cells and an effective amount of a foggy polypeptide antagonist.
